(19) United States
(12) Patent Application Publication
(10) Pub. No.: US 2023/0272353 A1
(43) Pub. Date: Aug. 31, 2023

(54) Long Non-Coding RNA-Expression In Fungal Hosts

(71) Applicant: Novozymes A/S, Bagsvaerd (DK)

(72) Inventors: Debbie Yaver, Davis, CA (US); Astrid Mach-Aigner, Wien (AT); Robert Mach, Wien (AT); Petra Till, Wien (AT)

(73) Assignee: Novozymes A/S, Bagsvaerd (DK)

(21) Appl. No.: 16/971,174

(22) PCT Filed: Feb. 21, 2019
(86) PCT No.: PCT/US19/18942

§ 371 Date: Aug. 19, 2020

Related U.S. Application Data
(60) Provisional application No. 62/634,260, filed on Feb. 23, 2018.
(60) Provisional application No. 62/673,188, filed on May 18, 2018.
(60) Provisional application No. 62/713,153, filed on Aug. 1, 2018.

(51) Int. Cl. C12N 9/02; C12N 15/113; C12N 15/80

(52) U.S. Cl. CPC C12N 9/0061; C12N 15/1137; C12Y 302/01004; C12N 15/80; C12N 2800/10

(57) ABSTRACT

The instant invention relates to a filamentous fungal host cell producing a polypeptide of interest, said host cell comprising one or more native or heterologous polynucleotide encoding a long non-coding RNA (lncRNA), wherein said lncRNA comprises more than 262 nucleotides and comprises a contiguous nucleotide sequence at its 3′ end, absent any poly-adenylation tail, that is at least 70% identical to that of SEQ ID NO:8, SEQ ID NO:9, SEQ ID NO: 10, SEQ ID NO: 19, SEQ ID NO:20 or SEQ ID NO:21; and/or said lncRNA comprises 2 or more Xyr1 binding sequences, as well as methods of producing a polypeptide of interest in said host cells and methods of improving the production of a polypeptide of interest.

### REFERENCE TO SEQUENCE LISTING

This application contains a Sequence Listing in computer readable form. The computer readable form is incorporated herein by reference.

### FIELD OF THE INVENTION

The present invention relates to filamentous fungal host cells producing one or more polypeptide of interest and expressing one or more long non-coding RNA (lncRNA-expression) in said host cells in order to improve the production, productivity and/or yield of said polypeptide of interest, as well as methods of producing the polypeptide of interest in said host cells.

### BACKGROUND OF THE INVENTION

Filamentous fungal host cells are widely employed for the industrial production of a wide variety of polypeptides of interest. Intense research efforts are directed at improving the production of polypeptides of interest in filamentous fungal host cells, especially into improving productivity and/or yield.

One example of this was reported in Austrian patent application (AT 509050 A4; published 15 Jun. 2011) which disclosed the alleged identification in Trichoderma reesei of a new putative hydrolase production activator termed Hax1. The predicted hax1 coding sequence and its encoded amino acid sequence were both provided, and the putative isolated hax1 nucleic acid and its heterologous expression was claimed. It was suggested, that the overexpression of hax1 would be useful, for example, for the culturing of Trichoderma reesei, e.g., for producing enzymes.

However, as it turns out, the predicted Trichoderma reesei hax1 open reading frame does not, in fact, encode a polypeptide.

### SUMMARY OF THE INVENTION

We show herein, that a so-called long non-coding RNA (lncRNA) is encoded by and transcribed from a genomic region in Trichoderma reesei beginning as far as 430 bp upstream of the erroneously predicted hax1 start-codon. The length of the 5′ end of the lncRNA turns out to be variable, but its 3′ end overlaps precisely with only the first 172 nucleotides of the 636 bp so-called hax1 open reading frame (which was disclosed as SEQ ID NO:3 in AT 509050 A4).

The inventors of the instant application have demonstrated in the examples below, that this lncRNA is an effective hydrolase activator in T. reesei, where its expression in varying lengths was able to improve cellulase production—as determined by cellulase activity.

Accordingly, in a first aspect, the invention provides filamentous fungal host cells producing a polypeptide of interest, said host cells comprising:
a) at least one polynucleotide encoding the polypeptide of interest;
b) one or more native or heterologous polynucleotide encoding a long non-coding RNA (lncRNA), wherein:
   i) said lncRNA comprises more than 262 nucleotides; preferably 299 nucleotides or more, 308 nucleotides or more, 309 nucleotides or more, or even 602 nucleotides or more, and said lncRNA comprises a contiguous nucleotide sequence at its 3′ end, absent any poly-adenylation tail, that is at least 70% identical to that of SEQ ID NO:8, SEQ ID NO:9, SEQ ID NO:10, SEQ ID NO:19, SEQ ID NO:20 or SEQ ID NO:21; preferably at least 75%, 80%, 85%, 90%, 95%, 96%, 97%, 98%, 99% or at least 100% identical to that of SEQ ID NO:8, SEQ ID NO:9, SEQ ID NO:10, SEQ ID NO:19, SEQ ID NO:20 or SEQ ID NO:21; and/or
   ii) said lncRNA comprises 2 or more Xyr1 binding sequences;

and wherein said one or more native or heterologous polynucleotide is fused with and operably linked to a heterologous promoter, whereby the one or more polynucleotide is transcribed into the lncRNA.

A second aspect of the invention relates to methods of producing a polypeptide of interest in a filamentous fungal host, comprising the steps of:
a) cultivating a filamentous fungal host cell as defined in any preceding claim under conditions conducive to the production of the polypeptide of interest; and, optionally
b) recovering the polypeptide of interest.

A final aspect of the invention relates to methods of improving the production, the productivity and/or the yield of a polypeptide of interest in a filamentous fungal host cell, comprising the steps of:
a) providing a filamentous fungal host cell comprising at least one polynucleotide encoding the polypeptide of interest; and
b) modifying the host cell to comprise one or more native or heterologous polynucleotide encoding a long non-coding RNA (lncRNA), wherein:
   i) said lncRNA comprises more than 262 nucleotides; preferably 299 nucleotides or more, 308 nucleotides or more, 309 nucleotides or more, or even 602 nucleotides or more, and said lncRNA comprises a contiguous nucleotide sequence at its 3′ end, absent any poly-adenylation tail, that is at least 70% identical to that of SEQ ID NO:8, SEQ ID NO:9, SEQ ID NO:10, SEQ ID NO:19, SEQ ID NO:20 or SEQ ID NO:21; preferably at least 75%, 80%, 85%, 90%, 95%, 96%, 97%, 98%, 99% or at least 100% identical to that of SEQ ID NO:8, SEQ ID NO:9, SEQ ID NO:10, SEQ ID NO:19, SEQ ID NO:20 or SEQ ID NO:21; and/or
   ii) said lncRNA comprises 2 or more Xyr1 binding sequences;

and wherein said one or more polynucleotide is fused with and operably linked to a heterologous promoter, whereby the one or more polynucleotide is transcribed into the lncRNA;

wherein the production, the productivity and/or the yield of the polypeptide of interest is improved in the modified host cell compared to its non-modified parent.

### BRIEF DESCRIPTION OF DRAWINGS

FIG. 1 shows a graphic representation of an in-silico prediction of a minimized free energy RNA secondary structure of the major lncRNA from T. reesei QM6a based on the RACE-defined 3′-and 5′-ends (see examples 2 and 3; SEQ ID NO:8).

FIG. 2 shows a graphic representation of an in-silico prediction of a centroid RNA secondary structure of the major lncRNA from T. reesei QM6a based on the RACE-defined 3′- and 5′-ends (see example 2).

FIG. 3 shows a graphic representation of an in-silico prediction of a mountain plot of the major lncRNA from T. reesei QM6a based on the RACE-defined 3′- and 5′-ends (see example 2).

FIG. 4 shows a graphic representation of an in-silico prediction of a minimized free energy RNA secondary structure of the major lncRNA from T. reesei QM9414 based on the RACE-defined 3′- and 5′-ends (see examples 2 and 3; SEQ ID NO:9).

FIG. 5 shows a graphic representation of an in-silico prediction of a centroid RNA secondary structure of the major lncRNA from T. reesei QM9414 based on the RACE-defined 3′- and 5′-ends (see example 2).

FIG. 6 shows a graphic representation of an in-silico prediction of a mountain plot of the major lncRNA from T. reesei QM9414 based on the RACE-defined 3′- and 5′-ends (see example 2).

FIG. 7 shows a graphic representation of an in-silico prediction of a minimized free energy RNA secondary structure of the major lncRNA from T. reesei Rut-C30 based on the RACE-defined 3′- and 5′-ends (see examples 2 and 3; SEQ ID NO:10).

FIG. 8 shows a graphic representation of an in-silico prediction of a centroid RNA secondary structure of the major lncRNA from T. reesei Rut-C30 based on the RACE-defined 3′- and 5′-ends (see example 2).

FIG. 9 shows a graphic representation of an in-silico prediction of a mountain plot of the major lncRNA from T. reesei Rut-C30 based on the RACE-defined 3′- and 5′-ends (see example 2).

FIG. 10 shows a graphic representation of an in-silico prediction of a minimized free energy RNA secondary structure of the major lncRNA from T. reesei Rut-C30 (5′-119), (see example 4; SEQ ID NO:19).

FIG. 11 shows a graphic representation of an in-silico prediction of a minimized free energy RNA secondary structure of the major lncRNA from T. reesei Rut-C30 (5′-120), (see example 4; SEQ ID NO:21).

FIG. 12 shows a graphic representation of an in-silico prediction of a minimized free energy RNA secondary structure of the major lncRNA from T. reesei Rut-C30 (5′+174), (see example 4; SEQ ID NO:20).

### DEFINITIONS

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Long non-coding RNA: A “long non-coding RNA” or “lncRNA” is a transcript longer than 200 nucleotides that is not translated into protein.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Xyr1 Regulatory Element or XRE: A 12 bp-long, palindromic DNA-motive in the xyr1 promoter (Pxyr1) that has been identified as a potential regulatory element of xyr1 expression, shown in SEQ ID NO:37.

Xyr1 Binding Sequence or XBS: Recognition sites for Xyr1, the key regulator of cellulase-and xylanase expression in T. reesei. The XBS sequence motif GGCWWW (SEQ ID NO:11) is the classical recognition site for the key-regulator of cellulase- and xylanase expression in T. reesei: Xyr1 (Furukawa, T. et al. Identification of specific binding sites for XYR1, a transcriptional activator of cellulolytic and xylanolytic genes in Trichoderma reesei. Fungal Genet. Biol. 46, 564-74 (2009)). The three first nucleic acids in the classic XBS motif: GGC, is the pivotal element to allow binding of Xyr1, even in the presence of at least one nucleic acid mismatch in the three WWW nucleic acids of the classic XBS motif, it is still considered a functional Xyr1 binding site; preferably the Xyr1 binding sequences of the instant invention comprise one or more XBS having the nucleotide sequence shown in SEQ ID NO:11, wherein at least one of the three A or T (denoted W) nucleic acid residues may be substituted for another nucleic acid; and/or one or more XRE having the nucleotide sequence shown in SEQ ID NO:37.

Sequence identity: The relatedness between two amino acid sequences or between two nucleotide sequences is described by the parameter “sequence identity”.

For the purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide or ribonucleotide sequences is determined using the sequences without any poly-adenylation tail they may have, and using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows: (Identical Deoxyribonucleotides x 100)/(Length of Alignment—Total Number of Gaps in Alignment)

### DETAILED DESCRIPTION OF THE INVENTION

### Host Cells

The present invention relates to recombinant host cells, comprising a polynucleotide of the present invention operably linked to one or more control sequences that direct the production of a polypeptide of interest. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The fungal host cell is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787.

A first aspect of the invention relates to filamentous fungal host cells producing a polypeptide of interest, said host cells comprising:
a) at least one polynucleotide encoding the polypeptide of interest;
b) one or more native or heterologous polynucleotide encoding a long non-coding RNA (lncRNA), wherein:
   i) said lncRNA comprises more than 262 nucleotides; preferably 299 nucleotides or more, 308 nucleotides or more, 309 nucleotides or more, or even 602 nucleotides or more, and said lncRNA comprises a contiguous nucleotide sequence at its 3′ end, absent any poly-adenylation tail, that is at least 70% identical to that of SEQ ID NO:8, SEQ ID NO:9, SEQ ID NO:10, SEQ ID NO:19, SEQ ID NO:20 or SEQ ID NO:21; preferably at least 75%, 80%, 85%, 90%, 95%, 96%, 97%, 98%, 99% or at least 100% identical to that of SEQ ID NO:8, SEQ ID NO:9, SEQ ID NO:10, SEQ ID NO:19, SEQ ID NO:20 or SEQ ID NO:21; and/or
   ii) said lncRNA comprises 2 or more Xyr1 binding sequences;

and wherein said one or more native or heterologous polynucleotide is fused with and operably linked to a heterologous promoter, whereby the one or more polynucleotide is transcribed into the lncRNA.

In a preferred embodiment of the invention, the filamentous fungal host cell is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phiebia, Piromyces, Pleurotus, Schizophyllum, Talaromyces, Thermoascus, Thielavia, Tolypocladium, Trametes, or Trichoderma cell; preferably the host cell is an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium suiphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phiebia radiata, Pleurotus eryngii, Thielavia terrestris, Trametes villosa, Trametes versicolor, Trichoderma harzianum, Trichoderma koningii, Trichoderma Iongibrachiatum, Trichoderma reesei, or Trichoderma viride cell; most preferably the host cell is a Trichoderma reesei cell.

In a preferred embodiment of the invention, the polypeptide of interest is native or heterologous to the host cell; preferably the native or heterologous polypeptide is a secreted polypeptide. It is also preferable that the polypeptide of interest is a hormone, enzyme, receptor or portion thereof, antibody or portion thereof; preferably the polypeptide of interest is an enzyme; even more preferably a hydrolase, isomerase, ligase, lyase, oxidoreductase, or transferase; still more preferably an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

It is preferable, in the aspects of the invention, that the lncRNA, absent any poly-adenylation tail, comprises or consists of a nucleotide sequence that is at least 70% identical to that of SEQ ID NO:8, SEQ ID NO:9, SEQ ID NO:10, SEQ ID NO:19, SEQ ID NO:20 or SEQ ID NO:21; preferably at least 75%, 80%, 85%, 90%, 95%, 96%, 97%, 98%, 99% or at least 100% identical to that of SEQ ID NO:8, SEQ ID NO:9, SEQ ID NO:10, SEQ ID NO:19, SEQ ID NO:20 or SEQ ID NO:21.

It is also preferable, in the aspects of the invention, that the lncRNA comprises 3 or more Xyr1 binding sequences; preferably 4, 5, 6, 7, 8, 9, 10, 11, 12, 13, 14, 15, 16, 17, 18, 19 or 20 or more Xyr1 binding sequences; preferably the Xyr1 binding sequences comprise one or more XBS having the nucleotide sequence shown in SEQ ID NO:11, wherein at least one of the three A or T (denoted W) nucleic acid residues may be substituted for another nucleic acid; and/or one or more XRE having the nucleotide sequence shown in SEQ ID NO:37.

Preferably, in the aspects of the invention, the heterologous promoter is a constitutive or an inducible promoter; preferably the promoter is obtained from the genes for Aspergillus nidulans acetamidase, Aspergillus niger neutral alpha-amylase, Aspergillus niger acid stable alpha-amylase, Aspergillus niger or Aspergillus awamori glucoamylase, Aspergillus oryzae TAKA amylase, Aspergillus oryzae alkaline protease, Aspergillus oryzae triose phosphate isomerase, Fusarium oxysporum trypsin-like protease, Fusarium venenatum amyloglucosidase, Fusarium venenatum Daria, Fusarium venenatum Quinn, Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase Ill, Trichoderma reesei endoglucanase V, Trichoderma reesei xylanase I, Trichoderma reesei xylanase II, Trichoderma reesei xylanase Ill, Trichoderma reesei beta-xylosidase, or Trichoderma reesei translation elongation factor; most preferably the promoter is the Trichoderma reesei bgl1 promoter.

### Methods of Production

The present invention also relates to methods of producing a polypeptide of interest, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

A second aspect of the invention relates to methods of producing a polypeptide of interest in a filamentous fungal host, comprising the steps of:
c) cultivating a filamentous fungal host cell as defined in the first aspect under conditions conducive to the production of the polypeptide of interest; and, optionally
d) recovering the polypeptide of interest.

A final aspect of the invention relates to methods of improving the production, the productivity and/or the yield of a polypeptide of interest in a filamentous fungal host cell, comprising the steps of:
c) providing a filamentous fungal host cell comprising at least one polynucleotide encoding the polypeptide of interest; and
d) modifying the host cell to comprise one or more native or heterologous polynucleotide encoding a long non-coding RNA (lncRNA), wherein:
   i) said lncRNA comprises more than 262 nucleotides; preferably 299 nucleotides or more, 308 nucleotides or more, 309 nucleotides or more, or even 602 nucleotides or more, and said lncRNA comprises a contiguous nucleotide sequence at its 3′ end, absent any poly-adenylation tail, that is at least 70% identical to that of SEQ ID NO:8, SEQ ID NO:9, SEQ ID NO:10, SEQ ID NO:19, SEQ ID NO:20 or SEQ ID NO:21; preferably at least 75%, 80%, 85%, 90%, 95%, 96%, 97%, 98%, 99% or at least 100% identical to that of SEQ ID NO:8, SEQ ID NO:9, SEQ ID NO:10, SEQ ID NO:19, SEQ ID NO:20 or SEQ ID NO:21; and/or
   ii) said lncRNA comprises 2 or more Xyr1 binding sequences;
and wherein said one or more polynucleotide is fused with and operably linked to a heterologous promoter, whereby the one or more polynucleotide is transcribed into the lncRNA;
   wherein the production, the productivity and/or the yield of the polypeptide of interest is improved in the modified host cell compared to its non-modified parent.

### Examples

### Strains

Trichoderma reesei QM6a is the commercially available ATCC 13631 wild-type strain.

Trichoderma reesei QM6a_Δtmus53 is a human LIG4 homolog strain having a higher efficiency of transformation compared to the wild-type. It is described in Steiger MG, Vitikainen M, Uskonen P, Brunner K, Adam G, Pakula T, Penttils M, Saloheimo M, Mach RL, Mach-Aigner AR. (2011) Transformation system for Hypocrea jecorina (Trichoderma reesei) that favors homologous integration and employs reusable bidirectionally selectable markers. AppI Environ Microbiol 77(1):114-21 doi:AEM.02100-10 fpii].

Trichoderma reesei QM6a_Δtmus53_Δpyr4 (abbreviation QM6a_Δpyr4) was derived from QM6a_Δtmus53 and is featured by uridine auxotrophy and resistance to 5-fluoorotic acid (5-FOA) due to the deletion of pyr4 (orotidine 5′-phosphate decarboxylase encoding gene). It is described in Derntl C, Kiesenhofer DP, Mach RL, Mach-Aigner AR (2015) Novel strategies for genomic manipulation of Trichoderma reesei with the purpose of strain engineering. AppI Environ Microbiol 81(18):6314-23 doi:10.1128/AEM.01545-15.

Trichoderma reesei QM6a_Δtmus53_Δpyr4 (LoxP) (abbreviation QM6a_LoxP) was derived from QM6a_Δtmus53 and is featured by uridine auxotrophy and resistance to 5-FOA due to the integration of Cre recombinase-encoding gene at the pyr4 locus. It is described in Steiger MG, Vitikainen M, Uskonen P, Brunner K, Adam G, Pakula T, Penttils M, Saloheimo M, Mach RL, Mach-Aigner AR. (2011) Transformation system for Hypocrea jecorina (Trichoderma reesei) that favors homologous integration and employs reusable bidirectionally selectable markers. AppI Environ Microbiol 77(1):114-21 doi:AEM.02100-10 fpii].

Trichoderma reesei QM6a_Δtmus53_Δxyr1 (abbreviation QM6a_Δxyr1) was derived from QM6a_Δtmus53 and bears a deletion of Xyr1, the main trans-activator of cellulase and xylanase encoding genes. It is described in Mello-de-Sousa™, RassingerA, Pucher ME, dos Santos Castro L, Persinoti GF, Silva-Rocha R, Pocas-Fonseca MJ, Mach RL, Nascimento Silva R, Mach-AignerAR (2015) The impact of chromatin remodelling on cellulase expression in Trichoderma reesei. BMC Genomics 16:588 doi:10.1186/s12864-015-1807-7.

Trichoderma reesei QM9414 was derived from QM6a by random mutagenesis and selection for increased cellulase- and xylanase production. It is commercially available as ATCC 26921.

Trichoderma reesei Rut-C30 was likewise derived from QM6a by random mutagenesis and selection for increased cellulase- and xylanase production, it is featured by a partial release from carbon catabolite repression and is the progenitor of industrial strains. It is commercially available as ATCC 56765.

### Media and Solutions

Buffer P was composed of 39.63 g (NH₄)₂SO₄ (Sigma), 2.85 g KH₂PO₄ and 0.71 g K₂HPO₄ dissolved in deionized water and autoclaved.

0.2 M Citric acid was composed of 42 g of citric acid, dissolved in deionized water to 1 liter and autoclaved.

DNA extraction buffer was composed of 0.1 M Tris/HCl (pH 8.0), 1.2 M NaCl and 5 mM EDTA dissolved in deionized water.

EMSA buffer was composed of 10 mM Tricine and 50 mM NaCl dissolved in deionized water. The pH was adjusted to 7.4. For RNA-applications the buffer was DEPC-treated. 100 mg/ml 5-FOA stock solution was composed of 1 g 5-fluoorotic acid dissolved in 10 ml DMSO

LB medium was composed of 10 g of tryptone, 5 g of yeast extract, 5 g of sodium chloride, dissolved in deionized water to 1 liter and autoclaved. For casting plates 1.5% agar were added before autoclaving.

50x MA trace elements solution was composed of 250 mg of FeSO₄-7H₂0, 85 mg of MnSO₄—H₂O, 70 mg of ZnSO₄-7H₂0, 100 mg of CaCI2-2H₂0 dissolved in deionized water. The pH was adjusted to 2.0, the solution was filled up to 1 liter and autoclaved.

Malt extract (MEX) was composed of 3% (w/v) malt extract and 0.1% peptone (w/v); dissolved in tap water. For casting plates 1.5% agar were added before autoclaving.

Mandels-Andreotti (MA) medium was composed of 20 ml of 50x MA trace elements solution, 250 ml 2x Mineral salt solution, 480 ml 0.1 M Phosphate-citrate buffer, 1 ml 5 M urea and filled to 1 liter with deionized water and autoclaved. For casting plates 1.5% agar were added before autoclaving.

2x Mineral salt solution was composed of 5.6 g of (NH₄)₂SO₄, 8.0 g of KH₂PO₄, 1.2 g of MgSO₄-7H₂0, 1.6 g of CaCI2-2H₂0 dissolved in deionized water to 1 liter and autoclaved.

0.1 M Phosphate-citrate buffer was composed of 17.8 g of Na₂HPO₄-2H₂0, 10.5 g of CsH₈07 dissolved in deionized water. The pH was adjusted to 5.0, the solution was filled up to 1 liter and autoclaved.

1 M Na-Phosphate buffer (pH 5.8) was composed of 7.9 ml 1M Na₂HPO₄ and 92.1 ml 1M NaH₂PO₄.

111 mM Na-Phosphate buffer (pH 5.8) was composed of 11.1 ml of the 1 M Na-Phosphate buffer and 88.9 ml deionized water. From this buffer, solutions of 2.5 mM ABTS, 630 mM D-glucose or 1U/ml HRP were prepared and applied for the GoxA assays.

Protein binding buffer was composed of 0.5 M NaCl, 20 mM Tris-HCl and 5 mM imidazole dissolved in deionized water. The pH was adjusted to 7.9

Protein elution buffer (modified) was composed of 0.5 M NaCl, 20 mM Tris-HCl, 120 mM imidazole dissolved in deionized water. The pH was adjusted to 7.9

PEG solution was composed of 12.47 g PEG 6000 (Sigma), 0.375 g CaCl₂.2H₂0 and 0.5 ml 1M Tris-HCl (pH 7.5) dissolved in Gibco water and sterile filtered.

RNA loading dye was composed of 95% ultrapure formamide, 0.025% bromphenol blue, 0.025% xylene cyanol FF and 5 mM EDTA dissolved in DECP-treated deionized water. The pH was adjusted to 8.0

STC buffer was composed of 43.6 g Sorbitol (Sigma), 0.29 g CaCl₂.2H₂0 and 2 ml 1M Tris-HCl (pH 7.4) dissolved in Gibco water. The pH was adjusted to 5.0, the solution was filled up to to 200 ml and sterile filtered.

TE buffer was composed of 10 mM Tris/HCl (pH 8.0) and 1 mM EDTA dissolved in deionized water. For RNA-applications the buffer was DEPC-treated.

TEN buffer was composed of 10 mM Tris/HCl (pH 8.0), 1 mM EDTA and 100 mM NaCl₂ dissolved in deionized water. For RNA-applications the buffer was DEPC-treated.

10x TBE was composed of 0.89 M Tris-HCl, 0.89 M boric acid (approximately 60 g per liter) and 0.02 M EDTA dissolved in deionized water. The pH was adjusted to 8.0 with boric acid. For RNA-applications the buffer was DEPC-treated.

### Example 1: Identification of Different Versions of a lncRNA Varying in Length in Moderate Cellulase Producing and Overproducing Trichoderma reesei Strains Via 3′ and 5′ Rapid Amplification of cDNA Ends (RACE)

To concretely define the boundaries of the lncRNA in Trichoderma reesei QM6a, QM9414 and Rut-C30 via RACE, total RNA was extracted from fungal mycelia of each strain as described previously (Mello-de-Sousa™, Gorsche R, Rassinger A, Pogas-Fonseca MJ, Mach RL, Mach-Aigner AR. A truncated form of the Carbon catabolite repressor 1 increases cellulase production in Trichoderma reesei. Biotechnol Biofuels. 2014; 7:129). 5′ and 3′ RACE were performed using the 5′/3′ RACE Kit, 2nd generation (Roche, Basel, Switzerland). For all PCRs the GoTaq G2 polymerase (Promega) was applied. The final PCR products were extracted from a gel, blunt-end ligated into pJET1.2 (Thermo Scientific) and analyzed by sequencing (Microsynth). The obtained sequences were aligned to the genome of T. reesei QM6a v2.0 accessible in the Joint Genome Institute's Trichoderma reesei genome database v. 2.0 at http://genome.jgi-psf.org/Trire2/Trire2.home.html.

### 5′ RACE:

5′ RACE was carried out according to manufacturer's instructions, applying 0.9-1 μg of DNase I digested RNA extracts for cDNA synthesis in a total reaction volume of 20 μl. For this initial reverse transcription step the gene specific primer rev_1.Intron (SEQ ID NO:1) was used.

After RNase A digestion and purification with the QiAquick PCR Purification Kit (Qiagen, Hilden, Germany) based on the modified protocol for RACE applications recommended by Roche, a poly(A)-tailing was performed. Subsequently, the PCR fragments were specifically amplified from the total pool with the Oligo dT-Anchor Primer (included in the kit) and rev_5′RACE_2 (SEQ ID NO:2) in a first PCR. The resulting product was diluted 1:50 and used as a template for a nested PCR with either rev up-Intron (SEQ ID NO:3) or rev_5′RACE_4 (SEQ ID NO:4) and the PCR Anchor primer included in the kit.

### 3′ RACE:

For 3′ RACE, the lncRNA-encoding sequence was enriched from 1.25-2.1 mg total RNA extract using a biotinylated and HPLC-purified specific DNA probe (sonde_5-Biotin; SEQ ID NO:5) as well as streptavidin-linked magnetic beads included in the μMACS™ Streptavidin Kit and the corresponding μMACS separator (Miltenyi Biotec, Bergisch Gladbach, Germany) based on the manufacturer's instructions. According to the unusual high calculated melting temperature of the biotinylated probe, initial denaturation was performed at 85° C. for 5 min and annealing to appropriate amounts of streptavidin-linked magnetic beads was done at 70° C. for 15 min. RNase-free TEN buffer and TE buffer were used for binding and washing, respectively. Enriched RNA was eluted with 150 μl RNase-free dH₂0 and digested with DNase I (Thermo Scientific). For cDNA synthesis, the RNA was purified using the GeneJET RNA Cleanup and Concentration Micro Kit (Thermo Scientific) as instructed in the protocol for purification of DNase I digested samples. The RNA was eluted from 4 columns with 10 μl each and pooled for cDNA synthesis. Reverse transcription with the Oligo dT-Anchor Primer was performed according to manufacturer's instructions. The derived cDNAs were RNase A digested and used for amplification of the lncRNA-encoding sequence applying the gene specific primers up-for_2 (SEQ ID NO:6) and for_3′RACE_3 (SEQ ID NO:7) for the initial and nested PCRs, respectively.

### Results:

In each of the three observed strains, i.e. QM6a, QM9414 and Rut-C30, 3′ RACE defined the major 3′-end of the lncRNA to the same nucleotide. However, the 5′-ends defined via 5′ RACE turned out to be different in the wildtype QM6a cellulase producing strain and the two cellulase overproducing strains. In QM6a, the most frequent 5′-end was found in transcripts of 262 nucleotides in length, the sequence of which is shown in SEQ ID NO:8.

In the cellulase overproducing strain QM9414, the obtained RNA transcript was found to be somewhat longer than in QM6a, a total of 299 nucleotides in length, the sequence is shown in SEQ ID NO:9. In both strains we also observed but, only rarely, longer versions of the RNA transcripts up to 323 nucleotides in length.

The highest abundance of long RNA transcripts was found in the high-yielding cellulase overproducing, Rut-C30. There, the major transcript was 428 nucleotides in length, as shown in SEQ ID NO:10. Some longer as well as shorter transcripts were also detected.

Besides the differences in RNA-length, another remarkable characteristic of the RNA transcripts is the presence of several recognition sites for Xyr1, the key regulator of cellulase- and xylanase expression in T. reesei. The XBS sequence motif GGCWWW (SEQ ID NO:11) is the classical recognition site for the key-regulator of cellulase- and xylanase expression in T. reesei: Xyr1 (Furukawa, T. et al. Identification of specific binding sites for XYR1, a transcriptional activator of cellulolytic and xylanolytic genes in Trichoderma reesei. Fungal Genet. Biol. 46, 564-74 (2009)). The three first nucleic acids in the classic XBS motif: GGC, is the pivotal element to allow binding of Xyr1, even in the presence of at least one nucleic acid mismatch in the three WWW nucleic acids of the classic XBS motif, it is still considered a functional Xyr1 binding site.

At the promoter regions of cellulase- and xylanase encoding genes, Xyr1 binds to XBS and thus causes transcriptional activation. The presence of several XBSs located close together nearby the transcriptional start site of the shortest transcript version points towards a physical interaction with the Xyr1 regulatory protein. All three versions of the lncRNA have 3 classical XBS and 2 XBS bearing one mismatch in the WWW of the XBS motive. In addition to those 5 XBS at the minus-strand encoded lncRNA, further XBS or such with one mismatch in the WWW of the XBS motive are present on the genome at the plus-strand. In sum, 7, 9 and 10 XBS or XBS bearing one mismatch are located on the genomic region of the lncRNA identified in QM6a, QM9414 and Rut-C30, respectively. This fact points towards a regulatory impact of Xyr1 on the production of the lncRNA and might allow distinct regulation of the different transcript versions.

The findings obtained from RACE and the lncRNA sequence properties clearly show that the lncRNA transcripts are different in the strains QM6a, QM9414 and Rut-C30; the function of the lncRNA may depend on 5′ transcript length and/or on the number of Xyr1 binding sequences vis-à-vis its interplay with the corresponding trans-activator, Xyr1. The results are summarized in table 1 below.

TABLE 1
Number of XBSs

| | | | | Number | |
|---|---|---|---|---|---|
| | | | | of XBS | Total |
| | | | Number of | with 1 | number |
| | | | ‘classic’ | mismatch | of XBS |
| | Cellulase | Transcript | XBS on | on the | on the |
| Strain | production | length | the RNA | RNA | genome |
| QM6a | Moderate | 262 nt | 3 | 2 | 7 |
| QM9414 | Overproduction | 299 nt | 3 | 2 | 9 |
| Rut-C30 | High-yield | 428 nt | 3 | 2 | 10 |
| | overproduction | | | | |

### Example 2: In Silico Structure Prediction of the lncRNA

In silico prediction of RNA secondary structures of the major lncRNAs from QM6a, QM9414 5 and Rut-C30, based on the RACE-defined 3′- and 5′-ends, was performed using the Vienna RNAfold Web server provided by the University of Vienna at http://rna.tbi.univie.ac.at/cgi-bin/RNAWebSuite/RNAfold.cgi. The Vienna RNA websuite was also disclosed in detail in Gruber et al. 2008, The Vienna RNA Websuite, W70-W74 Nucleic Acids Research, Vol. 36, Web Server issue. Structure prediction was based on minimized free energy (MFE structures) or minimized total base-pair distance (centroid structures). The structures were obtained as MFE plain structure drawings, centroid plain structure drawings and mountain plots. In the mountain plots, MFE structures (MFE), centroid structures (centroid) and the thermodynamic ensemble of RNA structures (pf) are depicted in a plot of height versus position, where the height m(k) is given by the number of base pairs enclosing the base at position k.

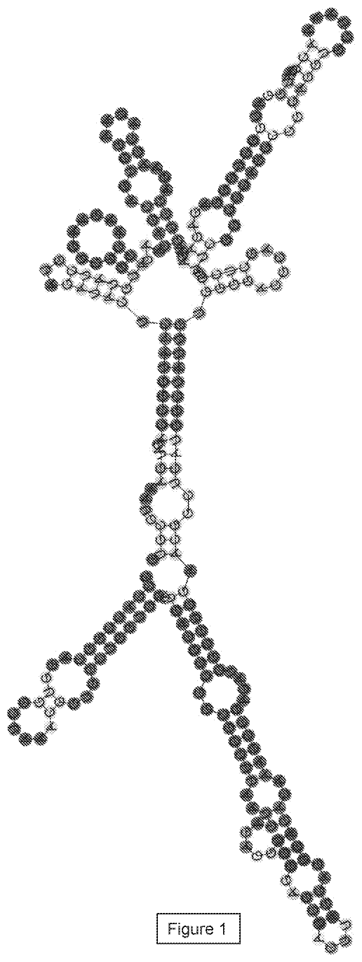

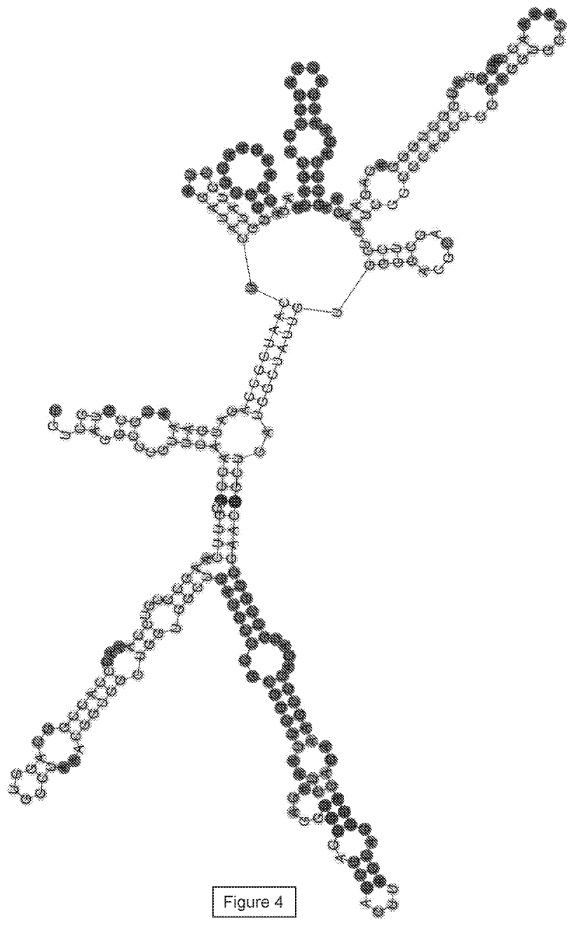

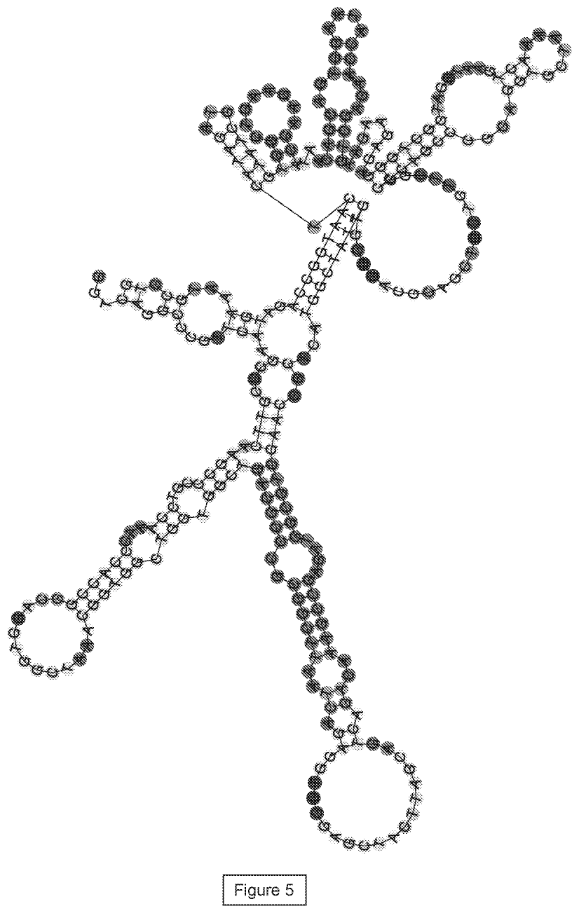

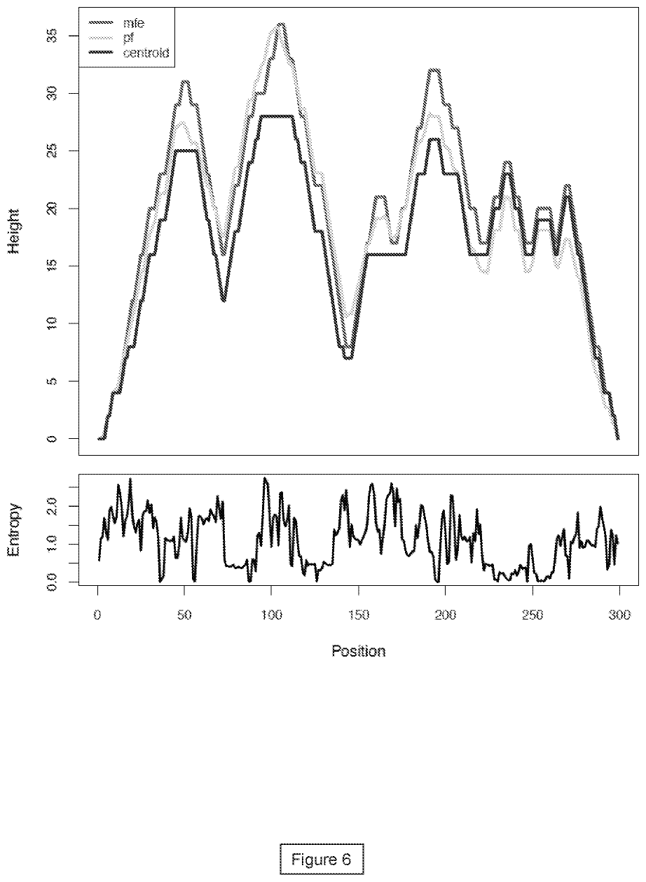

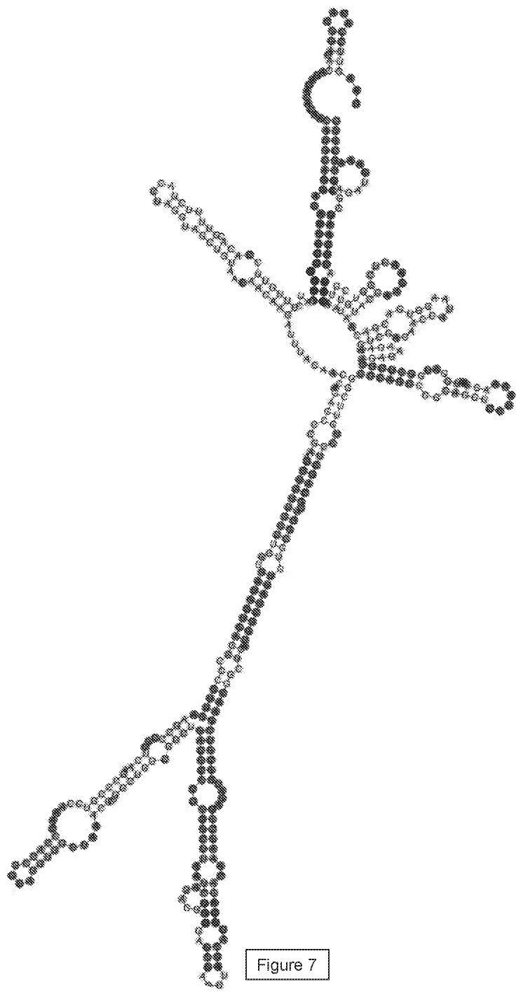

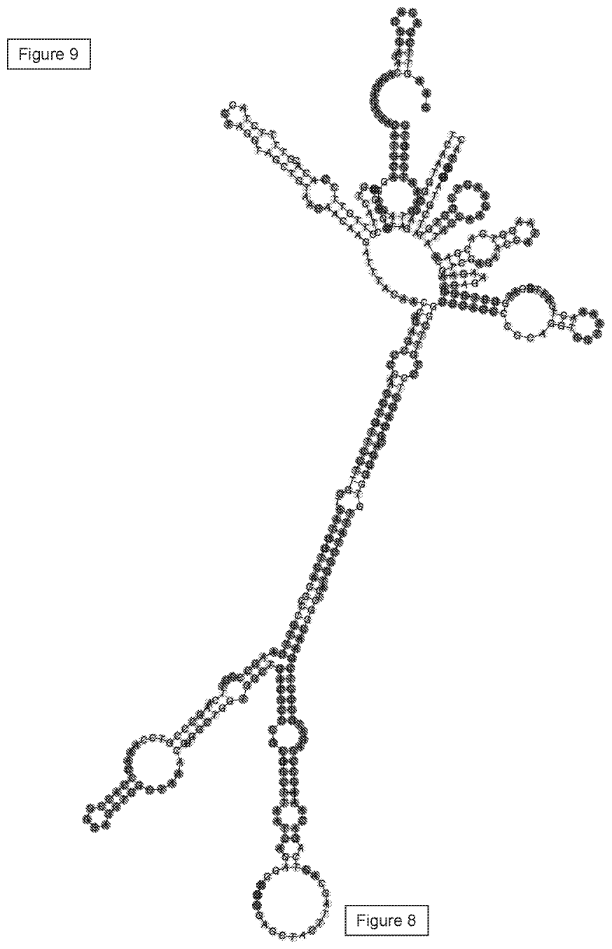

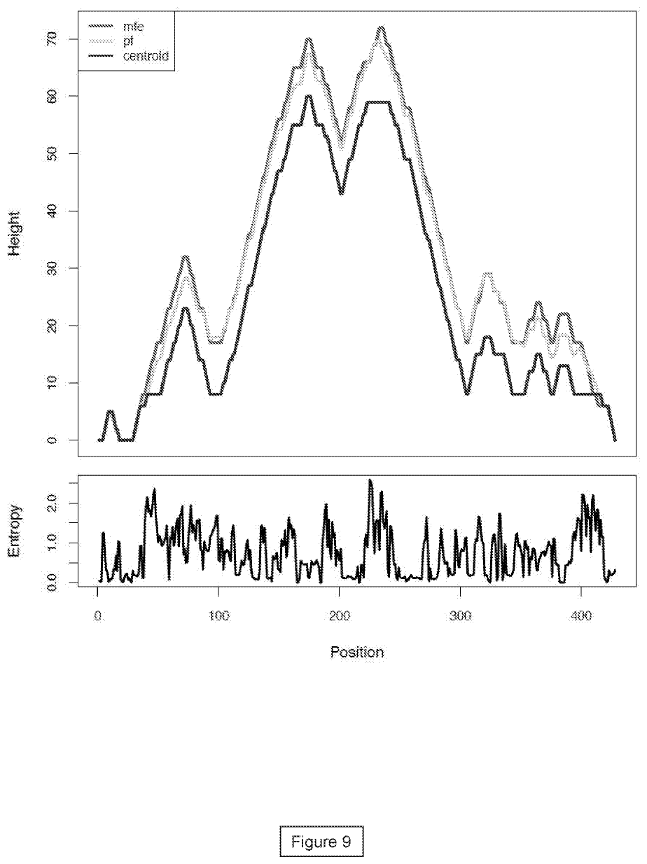

The obtained structures of are shown in FIGS. 1-3 (/ncRNA_{QM6a}), FIGS. 4-6 (/ncRNA_{QM9414}) and FIGS. 7-9 (/ncRNA_{Rut-c30}), respectively. Our studies indicated a 3′-polyadenylation of this lncRNA. However, the length or presence of a poly(A)-tail does not affect the predicted structures, it would only appear as an unpaired loop in MFE- and centroid plain structure drawings.

### Results

Based on the MFE plain structure drawings (FIGS. 1, 4, and 7), all versions of the lncRNAs are characterized by a typical formation of duplexed RNA. They are composed of a stem-forming and a hairpin-rich region at one end, and are split into two stem-loops at the other end. In the QM6a and QM9414 structures most of the hairpins are quite well conserved. However, in the QM9414 structure, an additional hairpin breaks the structure of the main stem. The Rut-C30 overall structure is rather different. In fact, the three structures only have one stem-loop in common. This supports the hypothesis that the 3 lncRNAs can act differently.

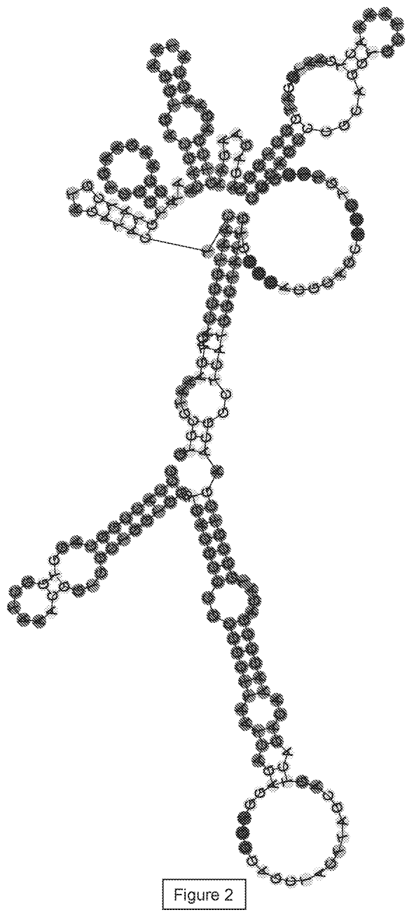

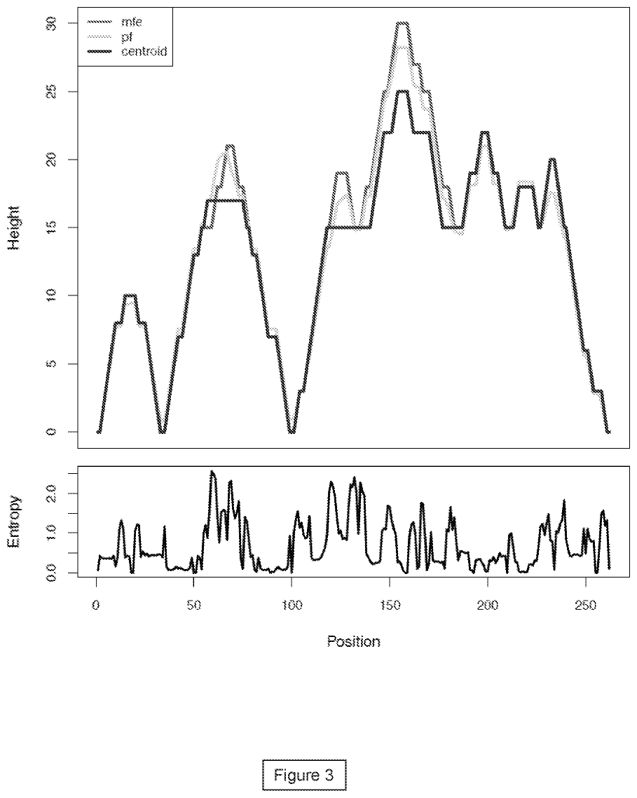

The same structural information can be inferred from centroid plain structure drawings (FIGS. 2, 5 and 8) and mountain plots (FIGS. 3, 6 and 9). In the mountain plots, hairpin loops appear as peaks and base-paired regions as valleys and slopes. The predicted RNA secondary structures are well represented by the displayed curves. Thus the validity of structural elements (i.e. stems and loops) predicted based on minimized free energy is supported.

### Example 3: Production and Analysis of T. reesei QM6a Strains Overexpressing the 3 Different lncRNA Versions

To learn about the impact of the three different versions of the lncRNA on cellulase production in T. reesei, the encoding DNA sequences were each fused with and operably linked to the bgl1 promoter, and ectopically integrated into the genome of T. reesei QM6a_Δtmus53, thereby deleting the pyr4 gene. The cellulase activities of the resulting lncRNA-overexpressing strains (termed OE strains) were assayed and compared to the parent and reference strains QM6a_Δtmus53 and QM6a _Δpyr4.

### Plasmid Construction:

For the generation of the lncRNA overexpression constructs, pCD-Δpyr4-Pbg|1-QM6A, pCD-Δpyr4-Pbg|1-QM9414 and pCD-Δpyr4-Pbgl1-Rut-C30, the respective lncRNA-encoding DNA segments were PCR amplified using chromosomal DNA of T. reesei QM6a as template. The following primers were used:
for_QM6a_Bcul (SEQ ID NO:12) and rev_3′QM6a (SEQ ID NO:13) for the 262 bp lncRNA from QM6a;
for_QM9414_Bcul (SEQ ID NO:14) and rev_3′QM6a (SEQ ID NO:13) for the 299 bp lncRNA from QM9414;
and for_Rut-C30_Bcul (SEQ ID NO:15) and rev_3′QM6a (SEQ ID NO:13)for the 428 bp lncRNA from Rut-C30.

The purified PCR products were blunt-end ligated into pJET1.2 (Thermo Scientific, Waltham, Mass., USA) to provide 3 pJET-lncRNA vectors, wherein the appropriate orientations were verified by digest with Bcul and Xbal.

In a next step a 997 bp fragment of the bgl1 promoter was PCR-amplified using the primers Pbgl1 for_Kpn2l (SEQ ID NO:16) and Pbgl1 rev-Nhel (SEQ ID NO:17), then digested with Kpn21 and Nhel, and subsequently cloned into the respective pJET-lncRNA vectors digested with Kpn21 and Bcul.

In order to prevent positioning of the lncRNA-encoding DNA sequence upstream of and the foreign 5′-untranslated region of the bgl1 promoter next to each other, the terminal point of the promoter fragment was chosen to be equal to the transcriptional start point previously defined for the bgl1 gene (Mach RL, Seiboth B, Myasnikov A, Gonzalez R, Strauss J, Harkki A M et al. The bgl1 gene of Trichoderma reesei QM 9414 encodes an extracellular, cellulose-inducible beta-glucosidase involved in cellulase induction by sophorose. Mol Microbiol. 1995; 16:687-697).

For construction of the final lncRNA overexpression cassettes, the Pbgl1-IncRNA fusion products were isolated from the plasmids by digestion with Kpn21 and Xbal, extracted from a gel, and introduced into Bcul/Kpn21-digested pCD-Δpyr4 carrying the cbh2 terminator (Derntl C, Kiesenhofer DP, Mach RL, Mach-Aigner AR. Novel Strategies for Genomic Manipulation of Trichoderma reesei with the Purpose of Strain Engineering. Appl Environ Microbiol. 2015; 81:6314-6323) in forward orientation.

For cloning of the constructs, Escherichia coli strain Top10 (Invitrogen, Life Technologies, Paisley, UK) was used. It was maintained on LB supplemented with 100 μg/ml ampicillin or spectinomycin and grown at 37° C. All PCRs were performed applying peqGOLD Pwo DNA polymerase (PEQLAB, Biotechnologie, Erlangen, Germany) according to the manufacturer's instructions. Final constructs were verified by sequencing (Microsynth, Balgach, Switzerland).

### Fungal Transformation:

Protoplast transformation of T. reesei was performed essentially as described previously (Gruber F, Visser J, Kubicek CP, de Graaff LH. The development of a heterologous transformation system for the cellulolytic fungus Trichoderma reesei based on a pyrG-negative mutant strain. Curr Genet. 1990; 18:71-76). 200 μg of the Notl-digested construct pCD-Δpyr4-Pbgl1-QM6a, pCD-Δpyr4-Pbgl1-QM9414 or pCD-Δpyr4-Pbgl1-Rut-C30, were used for transformation of 107 protoplasts (in 150 μl) of QM6a_Δtmus53. Selection for pyr4 deleted transformants was performed on MEX agar containing 1.2 M sorbitol, 1.5 mg/ml 5-FOA and 5 mM uridine as described by Derntl and co-workers (Derntl C, Kiesenhofer DP, Mach RL, Mach-Aigner AR. Novel Strategies for Genomic Manipulation of Trichoderma reeseiwith the Purpose of Strain Engineering. Appl Environ Microbiol. 2015; 81:6314-6323). The plates were incubated at 30° C. for 3-7 days until colonies were visible.

### Genotypic Characterization:

For an initial identification of uridine auxotroph OE strains, candidates were grown on MA medium containing 1% glycerol as a carbon source without peptone or uridine. The OE candidates that were unable to grow under these conditions were tested by PCR using the primers 5pyr4_fwd(Bglll) (SEQ ID NO:18) and rev_3′QM6a (SEQ ID NO:13) and GoTaq G2 polymerase (Promega). Extraction of chromosomal DNA for candidate screening was performed as described by Gruber and co-workers (Gruber, F., Visser, J., Kubicek, C.P. & de Graaff, L. H. The development of a heterologous transformation system for the cellulolytic fungus Trichoderma reesei based on a pyrG-negative mutant strain. Curr. Genet. 18, 71-6 (1990)) and according to the adaptions described previously (Derntl C, Kiesenhofer DP, Mach RL, Mach-Aigner AR. Novel Strategies for Genomic Manipulation of Trichoderma reesei with the Purpose of Strain Engineering. Appl Environ Microbiol. 2015; 81:6314-6323.).

The transformations yielded one verified strain for the overexpression of each lncRNA version denoted OE_{QM6a}, OE_{QM9414} and OE_{Rut-c30}, respectively. The strains were maintained MEX containing 5 mM uridine or MEX containing 1.5 mg/ml 5-FOA and 5 mM uridine at 30° C.

### Cellulase Assay:

For cellulase assays, the three OE strains, the parent strain QM6a_Δtmus53 and the reference strain QM6a_Δpyr4 were incubated for 72 h at 30° C. and 180 rpm in 100 ml MA medium containing 1% (w/v) α-D-lactose inoculated with 109 conidia per liter (final concentration). The cultivation was carried out in biological triplicates. The cellulase activity in the supernatants of the grown cultures was assayed in 25 mM sodium acetate buffer pH 4.5 using Azo-Cellazyme C tablets (Megazyme, Wicklow, Ireland) as substrate, essentially according to the manufacturer's instructions. The reaction time was increased to 75 min to obtain detectable values for all strains and samples with higher cellulase activity were adjusted by dilution in order to enable comparison.

Cellulase activities were calculated from the absorbance at 590 nm for 10 min reaction time based on the equation mU=232.6*Abs+5 (Steiger M. Der Aktivator Xyr1 und seine Bedeutung für den Laktosemetabolismus von T. reesei. Diploma thesis, TU Wien. 2007) and referred to the biomass dry weight derived from incubation of the harvested mycelia at 80° C. for 24 h. One unit is defined as the amount of enzyme required to release 1 μmole of D-glucose reducing-sugar-equivalents per minute under the respective assay conditions.

The strains are listed below in table 2 and the final values are shown further down in table 3, the values are the means of the biological triplicates and relative to those of QM6a_Δtmus53.

TABLE 2

| Strain | Abbreviation | Selection |
|---|---|---|
| QM6a_Δtmus53 | | |
| QM6a_Δtmus53_Δpyr4 | QM6a_Δpyr4 | |
| QM6a_Δtmus53_Δpyr4_Pbgl1::IncRNA_{QM6a} | OE_{QM6a} | deletion of pyr4 |
| QM6a_Δtmus53_Δpyr4_Pbgl1::IncRNA_{QM9414} | OE_{QM9414} | deletion of pyr4 |
| QM6a_Δtmus53_Δpyr4_Pbgl1::IncRNA_{Rut-C30} | OE_{Rut-C30} | deletion of pyr4 |

### Results:

The cellulase activities of the three OE strains, QM6a_Δtmus53 and QM6a_Δpyr4 are given in the table below. In all OE strains the cellulase activities were higher compared to the parent and reference strain, thus indicating an improved cellulase expression, improved cellulase productivity and/or improved yield, as measured by cellulase activity, resulting generally from the increased levels of the lncRNA.

Remarkably, we also observed an effect resulting from overexpression of each specific lncRNA. Overexpression of the shortest QM6a lncRNA had less of an impact on the cellulase production than that of the QM9414 lncRNA. The highest impact on cellulase activity however was achieved by overexpression of the longest Rut-C30 lncRNA, providing a nine-fold increase in expression, productivity and/or yield compared to the reference strains, as measured by cellulase enzyme activity.

TABLE 3

| | | Cellulase activity | |
|---|---|---|---|
| Strain | IncRNA length | relative to QM6a | SEQ ID NO |
| QM6a_Δtmus53 | | 1.00 | |
| QM6a_Δpyr4 | | 0.85 | |
| OE_{QM6a} | 262 nt | 2.15 | 8 |
| OE_{QM9414} | 299 nt | 5.40 | 9 |
| OE_{Rut-C30} | 428 nt | 9.27 | 10 |

### Example 4. Investigations on the Dependence of Function on Length and Folding of the lncRNA by the Production and Analysis of T. reesei QM6a Strains Overexpressing Truncated or Elongated Versions of the Long Rut-C30 lncRNA

To learn about the dependence of its function on length and folding, one elongated version (5′ +174 nt, SEQ ID NO:19) and two truncated versions (5′-119 nt, SEQ ID NO:20; 5′-120 nt SEQ ID NO:21) of the Rut-C30 lncRNA were chosen for further investigations based on in silico structure predictions.

As described in Example 3, the coding sequences were fused to and operably linked with the bgl1 promoter, and ectopically integrated into the genome of T. reesei QM6a_Δpyr4, in this case resulting in a reconstitution of the pyr4 gene. The cellulase activities of the obtained OE strains was assayed compared to the parent and reference strains QM6a_Δpyr4 and QM6a_Δtmus53.

In silico prediction of RNA secondary structures was performed using the Vienna RNAfold Web server provided by the University of Vienna (vide supra).

### Plasmid Construction:

For the generation of the lncRNA overexpression constructs pCD-RPyr4T-Pbgl1-Rut-C30, pCD-RPyr4T-Pbg|1-(5′+174), pCD-RPyr4T-Pbg|1-(5′-119) and pCD-RPyr4T-Pbg|1-(5′+120), the 997 bp bgl1 promoter described in Example 3 was PCR-amplified using the primers Pbgl1 for_Kpn2l (SEQ ID NO:16) and Pbgl rev_Xbal (SEQ ID NO:22) and chromosomal DNA of T. reesei QM6a as a template. The purified PCR product was blunt-end ligated into pJET1.2 (Thermo Scientific, Waltham, Mass., USA) and the appropriate orientation was verified by digest with Xbal.

In a next step, the different/ncRNA versions were amplified from chromosomal DNA of T. reesei QM6a using the following primers:
for_RutC30_Xbal (SEQ ID NO:23) and rev_3′QM6a_Bcul-Ncol (SEQ ID NO:24) for the 428 bp (Rut-C30 lncRNA);
for_RutC30 5-plus 174nt Xbal (SEQ ID NO:25) and rev_3′QM6a_Bcul-Ncol (SEQ ID NO:24) for the 602 bp (Rut-C30 lncRNA+174 nucleotides 5′);
for_RutC30 5-minus 119nt_Xbal (SEQ ID NO:26) and rev_3′QM6a_Bcul-Ncol (SEQ ID NO:24) for the 309 bp (Rut-C30 lncRNA -119 nucleotides 5′);
for_RutC30 5-minus 120nt_Xbal (SEQ ID NO:27) and rev_3′QM6a_Bcul-Ncol (SEQ ID NO:24) for the 308 bp (Rut-C30 lncRNA -120 nucleotides 5′).

The PCR-amplified lncRNA-encoding DNA fragments were purified, digested with Xbal and Ncol, and subsequently cloned into pJET-Pbg|1 digested with Xbal and Ncol. For construction of the final/ncRNA overexpression cassettes, the Pbgl1-IncRNA fusion products were isolated from the plasmids by digestion with Kpn2l and Bcul, extracted from a gel, and introduced into Bcul/Kpn2l-digested pCD-RePyr4T carrying the cbh2 terminator (Derntl C, Kiesenhofer DP, Mach RL, Mach-Aigner AR. Novel Strategies for Genomic Manipulation of Trichoderma reesei with the Purpose of Strain Engineering. Appl Environ Microbiol. 2015; 81:6314-6323.) in forward orientation. For cloning of the constructs, Escherichia coli strain Top10 (Invitrogen, Life Technologies, Paisley, UK) was used. It was maintained on LB supplemented with 100 μg/ml ampicillin or spectinomycin and grown at 37° C. All PCRs were performed applying peqGOLD Pwo DNA polymerase (PEQLAB, Biotechnologie, Erlangen, Germany) according to the manufacturer's instructions. Final constructs were verified by sequencing (Microsynth, Balgach, Switzerland).

### Fungal Transformation:

Protoplast transformation of T. reesei was performed essentially as described previous (Gruber F, Visser J, Kubicek CP, de Graaff LH. The development of a heterologous transformation system for the cellulolytic fungus Trichoderma reesei based on a pyrG-negative mutant strain. Curr Genet. 1990; 18:71-76).

50 μg of the Notl-digested pCD-RPyr4T-Pbgl1-Rut-C30, pCD-RPyr4T-Pbgl1-(5′+174), pCD-RPyr4T-Pbgl1-(5′-119) and pCD-RPyr4T-Pbgl1-(5′+120) were used for transformation of 107 protoplasts (in 150 μl) of QM6a_Δpyr4. For selection of prototrophy, 500 μl of the transformation reaction mixture were added to 20 ml melted, 50° C. warm MA medium agar containing 1.2 M sorbitol and 1% (w/v) D-glucose. This mixture was poured into sterile petri dishes. After solidification the plates were incubated at 30° C. for 3 to 7 days until colonies were visible.

### Genotypic Characterization:

Homokaryotic candidate strains were generated by one or two rounds of vegetative spore propagation on MEX agar containing 0.1% IGEPAL CA-630 (Sigma Aldrich). Chromosomal DNA for candidate screening was extracted as described by Gruber and co-workers (Gruber, F., Visser, J., Kubicek, C. P. & de Graaff, L. H. The development of a heterologous transformation system for the cellulolytic fungus Trichoderma reesei based on a pyrG-negative mutant strain. Curr. Genet. 18, 71-6 (1990)) and according to the adaptions described previously (Derntl C, Kiesenhofer DP, Mach RL, Mach-Aigner AR. Novel Strategies for Genomic Manipulation of Trichoderma reesei with the Purpose of Strain Engineering. Appl Environ Microbiol. 2015; 81:6314-6323). A number of OE transformant strains were selected for each construct.

The OE strains were tested by PCR using the primers 5pyr4_fwd3 (SEQ ID NO:28) and Pbgl rev_Xbal (SEQ ID NO:22) (locus-specific PCR) or 5pyr4_fwd2 (SEQ ID NO:29) and Tpyr4_rev-Notl (SEQ ID NO:30) (wild-type specific PCR) and GoTaq G2 polymerase (Promega). In addition, the strains were verified by Southern blot analysis as described previously (Derntl C, Kiesenhofer DP, Mach RL, Mach-Aigner AR. Novel Strategies for Genomic Manipulation of Trichoderma reesei with the Purpose of Strain Engineering. Appl Environ Microbiol. 2015; 81:6314-6323). For this purpose, 30 μg chromosomal DNA of OE strains were digested with Ncol, resulting in a 3551 bp fragment specific for the wild-type and a 2248 bp fragment specific for overexpression of the lncRNA. The locus-specific biotinylated probe applied for hybridization was derived from a PCR using the primer pair pyr4_3fwd (SEQ ID NO:31) and Tpyr4_rev2 (SEQ ID NO:32) for amplification.

The transformation yielded four verified strains of OE-Rut-C30_Re (_11-6, _11-7, _Ill-12-1, _Ill-12-2) and three strains of each of OE-(5′+174)_Re (_Ill-2, _Ill-11, _Ill-12); OE-(5′-119)_Re (_Ill-3-2, _IV-7, _IV-10); and OE-(5′-120)_Re L1-1, _11-2, _11-3). The OE strains were maintained on MA medium agar containing 1% (w/v) D-glucose or MEX at 30° C.

### Cellulase Assay:

For cellulase assays, the four obtained strains of OE-Rut-C30_Re, the three strains of each of OE-(5′+174)_Re, OE-(5′-119)_Re and OE-(5′-120)_Re, as well as the parent strain QM6a_Δpyr4 and the reference strain QM6a_Δtmus53 were incubated for 48 h at 30° C. and 180 rpm in 100 ml MA medium containing 1% (w/v) α-D-lactose inoculated with 10¹ conidia per liter (final concentration).

The cellulase activity in the supernatants of the grown cultures was assayed in 25 mM sodium acetate buffer pH 4.5 using Azo-Cellazyme C tablets (Megazyme, Wicklow, Ireland) as substrate, essentially according to the manufacturer's instructions. The reaction time was increased to 60 min to obtain detectable values for all strains and samples with higher cellulase activity were adjusted by dilution in order to enable comparison. Cellulase activities were calculated from the absorbance at 590 nm for 10 min reaction time based on the equation mU=232.6*Abs+5 (Steiger M. Der Aktivator Xyr1 und seine Bedeutung fQr den Laktosemetabolismus von T. reesei. Diploma thesis, TU Wien. 2007) and referred to the biomass dry weight derived from incubation of the harvested mycelia at 80° C. for 24 h. One unit is defined as the amount of enzyme required to release 1 μmole of D-glucose reducing-sugar-equivalents per minute under the respective assay conditions. The final values are means of the biological replicates derived from the independently generated strains and are given relative to QM6a_Δtmus53.

TABLE 4
Strains used in this example.

| | | | Selection |
|---|---|---|---|
| Strain | Abbreviation | Candidates | marker |
| QM6a_Δtmus53 | | | |
| QM6a_Δtmus53_Δpyr4 | QM6a_Δpyr4 | | |
| QM6a_Δtmus53_Pbgl1::lncRNA-Rut-C30 | OE-Rut-C30_Re | II-6, II-7, III-12-1, | reconstitution |
| | | III-12-2 | of pyr4 |
| QM6a_Δtmus53_Pbgl1::lncRNA(5′ + 174) | OE-(5′ + 174)_Re | III-2, III-11, III-12 | reconstitution |
| | | | of pyr4 |
| QM6a_Δtmus53_Pbgl1::lncRNA(5′ − 119) | OE-(5′ − 119)_Re | III-3-2, IV-7, IV-10 | reconstitution |
| | | | of pyr4 |
| QM6a_Δtmus53_Pbgl1::lncRNA(5′ − 120) | OE-(5 − 120)_Re | II-1, II-2, II-3 | reconstitution |
| | | | of pyr4 |

### Results:

Interestingly, in silico structure prediction revealed that a truncated lncRNA version lacking 119 nucleotides in the 5′ end (compared to the 428 nucleotide lncRNA from Rut-C30) still has the overall structural properties of the lncRNA from Rut-C30, whereas the removal of just one more nucleotide, i.e. lacking 120 nucleotides in the 5′ end, results in a different RNA folding more similar to that of the lncRNA from QM9414.

The impact of the two truncated lncRNA variants, (5′-119) and (5′-120), on cellulase expression was investigated in order to gain knowledge about the dependence of the lncRNA function on length and folding.

An extended lncRNA variant was also designed and denoted (5′+174) due it having 174 nucleotides added to its 5′ end (compared to the 428 nucleotide lncRNA from Rut-C30). We wanted to see if a longer lncRNA variant (native or artificial) might have an even more stimulating effect on cellulase expression than the 428 nucleotide lncRNA from Rut-C30.

The upper boundary of RNA length was defined as the 428 nucleotide lncRNA from Rut-C30 elongated in the 5′ end by 174 nucleotides, in accordance with the end of the coding region of the upstream adjacent gene in T. reesei Rut-C30 encoding 2-isopropylmalate synthase. The predicted structure of lncRNA (5′+174) still contains the striking parts of the Rut-C_30 folding, however it is more complex, comprising additional hairpins, loops and bulges.

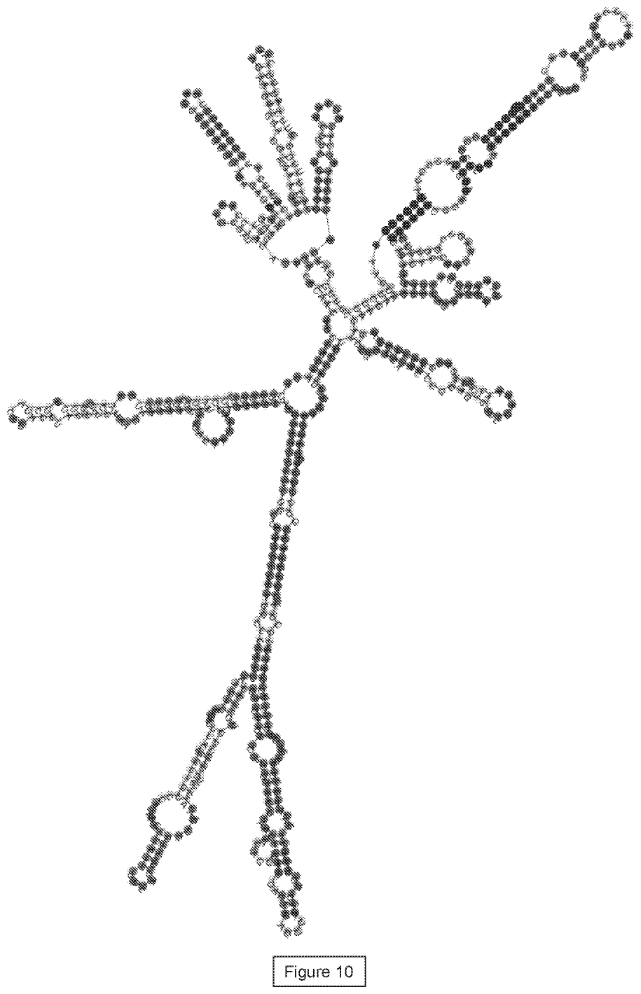

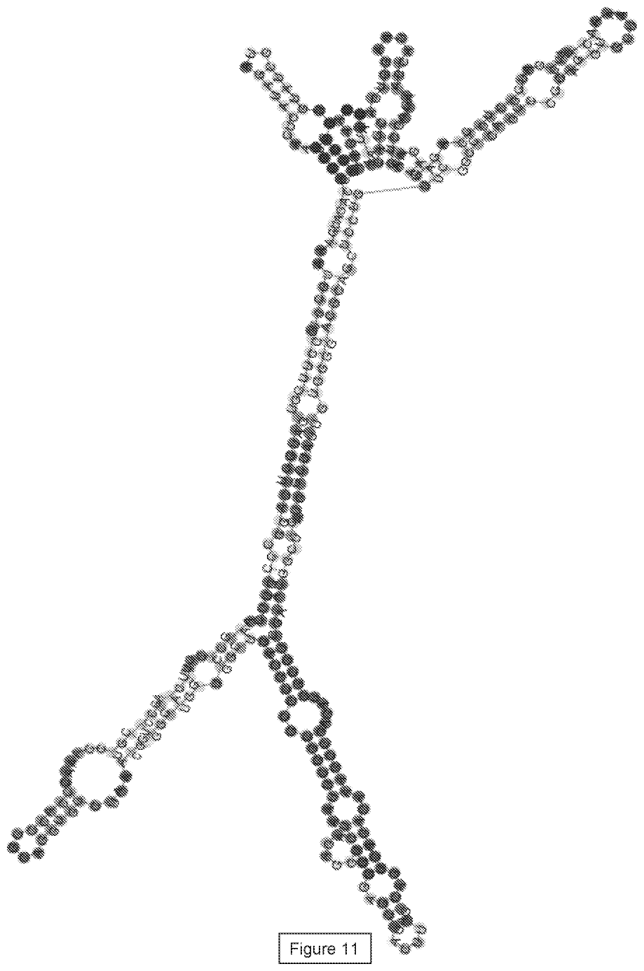

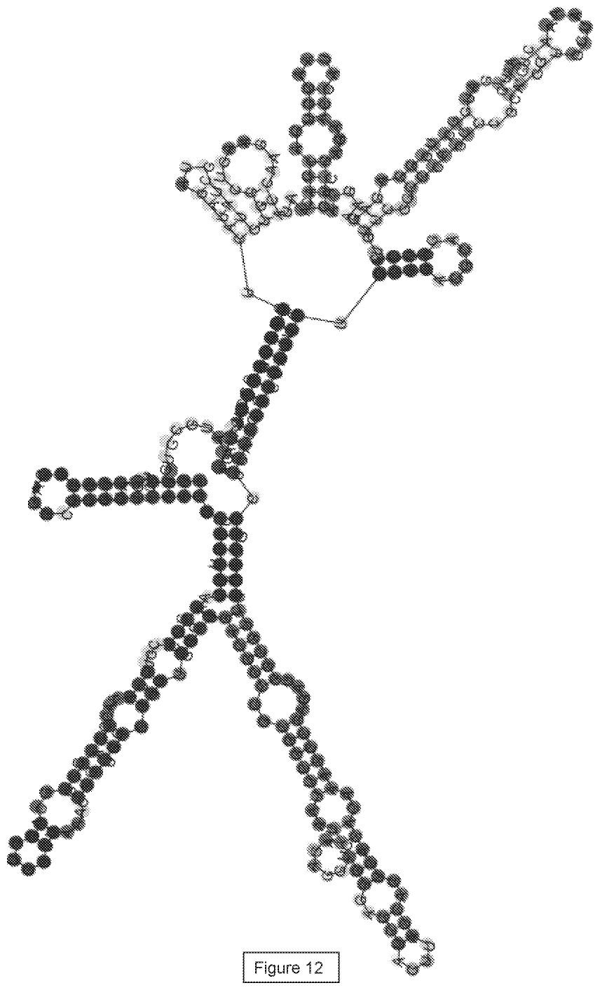

Graphical representations of the predicted structures of lncRNA (5′-119), (5′-120) and (5′+174) are shown in FIGS. 10-12, respectfully. The stability is increasing from light grey to dark grey As observed for the overexpression of the major lncRNA versions in a pyr4-deleted background (Example 3), the cellulase activities of all OE strains with a pyr4-reconstituted background are increased compared to the parent strain QM6a_Δpyr4 and the reference strain QM6a_Δtmus53. Results are shown in table 5 below.

However, the cellulase activity of OE-(5′+174)_Re is similar to that of OE-Rut-C30_Re, thus leading to the conclusion that no further functional improvement is likely to be achieved from merely increasing the length of the lncRNA with native Trichoderma reesei upstream sequence.

Furthermore, interesting conclusions regarding the connection of lncRNA length and folding can be drawn from comparing the two truncated versions (5′-119) and (5′-120). Though the former is only one single nucleotide longer than the latter, the cellulase activities of OE-(5′-1 19)_Re strains are much higher than those of OE-(5′-120)_Re strains, and even higher than in the parent strain OE-Rut-C30_Re. Yet, according to in silico structure prediction, the (5′-119) lncRNA folding resembles that of the Rut-C30 lncRNA, whereas that of (5′-120) is similar to that of QM9414.

Based on these results, we conclude that the function of the lncRNA in T. reesei depends on RNA folding rather than on RNA length. However, the folding of longer lncRNA versions has a more pronounced positive effect on cellulase expression than that of shorter versions.

TABLE 5

| | | Predicted | Cellulase activity |
|---|---|---|---|
| Strain | Length | folding like | relative to QM6a |
| QM6a_Δtmus53 | | | 1.00 |
| QM6a_Δpyr4 | | | 0.36 |
| OE-Rut-C30_Re | 428 nt | Rut-C30 | 2.61 |
| OE-(5′ + 174)_Re | 602 nt | Rut-C30/complex | 2.04 |
| OE-(5′ − 119)_Re | 309 nt | Rut-C30 | 3.99 |
| OE-(5′ − 120)_Re | 308 nt | QM9414 | 1.68 |

### Example 5: Electrophoretic Mobility Shift Assays (EMSAs) on the Protein-RNA Interaction of the Transactivator Xyr1 and the lncRNA

For lncRNA-Xyr1 interaction studies, the different versions of lncRNA (i.e. lncRNA_{QM6a}, IncRNA_{QM9414} and/ncRNA_{Rut-c30}) were in vitro synthesized and Xyr1 was heterologously expressed. Interactions were analyzed via RNA-EMSAs.

Preparation of Templates for the In Vitro Synthesis of lncRNA:

For the construction of pUC18-PT7-QM6a_{, pUC}18-PT7-QM9414 and pUC18-PT7-Rut-C30 for RNA in vitro synthesis, the T7-promoter was attached to the respective lncRNA encoding DNA polynucleotides via PCR with the primer pairs:
for_QM6a_PT7_Hindlll (SEQ ID NO:33) and rev_3′QM6a_Xbal (SEQ ID NO:34) for the QM6a lncRNA;
for_QM9414_PT7_Hindlll (SEQ ID NO:35) and rev_3′QM6a_Xbal (SEQ ID NO:34) for the QM9414 lncRNA;
for_RutC30_PT7_Hindlll (SEQ ID NO:36) and rev_3′QM6a_Xbal (SEQ ID NO:34) for the Rut-C30 lncRNA.

As a template, chromosomal DNA of T. reesei was used. Both, PT7-lncRNA and pUC18 were digested with Hindlll and Xbal, purified and ligated. Escherichia coli strain Top10 (Invitrogen, Life Technologies, Paisley, UK) was used for all cloning procedures. It was maintained on LB supplemented with 100 μg/ml ampicillin or spectinomycin and grown at 37° C. All PCRs were performed applying peqGOLD Pwo DNA polymerase (PEQLAB, Biotechnologie, Erlangen, Germany) according to the manufacturer's instructions.

In Vitro Synthesis of lncRNA:

In vitro synthesis of lncRNA was performed using the T7 High Yield RNA Synthesis Kit (New England Biolabs, Ipswich, Mass., USA) according to the manufacturer's instructions. For preparation of the templates for RNA in vitro synthesis, the plasmids pUC18-PT7-QM6a, pUC18-PT7-QM9414 and pUC18-PT7-Rut-C30 were linearized with Xbal (Thermo Scientific, Waltham, Mass., USA) downstream of the PT7-lncRNA insert, leaving 5′ overhangs.

The template DNA was purified by phenol/chloroform extraction, precipitated with 1/10 volume of 3 M sodium acetate and two volumes of ethanol and resuspended in 40 μl nuclease-free water. 1 μg template DNA was applied for standard RNA synthesis in a 20 μl reaction performed at 37° C. for 2 h. Subsequently, the synthesized RNA was DNasel digested at 37° C. for 15 min and again purified via phenol/chloroform extraction and precipitation with 1/10 volume of 3 M sodium acetate and 2 volumes of ethanol. Finally, the RNA-pellet was resolved in 50 μl nuclease-free water, quantified via Nano Drop and analyzed by denaturing polyacrylamide gel electrophoresis (PAGE).

For the denaturing PAGE, 0.5-1 μg lncRNA supplemented with 1.5 volumes RNA loading dye were heated at 95° C. for 5 min and separated on a 5% polyacrylamide gel (19:1 acrylamide:bis-acrylamide) containing 8 M urea at 15 mAmp for 45 min, essentially as described before (Rio, D. C., Ares Jr., M., Hannon, G. J., Nilsen, T. W. RNA: A Laboratory Manual, 59-63 (Cold Spring Harbor Laboratory Press, New York, 2011)).

### Expression and Purification of Xyr1:

300 mL LB medium with D-glucose (1% w/v) and kanamycin (50 μg/mL) were inoculated with E. coli BL21(DE3) (Promega) carrying the expression vector pTS1. At an ODeoₒ of 0.3, the protein expression was induced by adding IPTG to a final concentration of 0.5 mM. The culture was incubated at 18° C. for 24 h. The cells were harvested by centrifugation and stored frozen at −20° C. overnight. Cells were then resuspended in 10 mL Protein binding buffer and sonicated using a Sonifier® 250 Cell Disruptor (Branson, Danbury, USA) (power 40%, duty cycle 70%, power for 30 s, pause for 30 s, 410 cycles). After centrifugation, the protein (105 kDa) was purified from the extract using Novagen® HisBind® resin (Merck, Darmstadt, Germany) and Protein elution buffer (modified) according to manufacturer's guidelines. Finally, the buffer of the purified protein samples was exchanged to EMSA buffer applying PD-10 columns (GE Healthcare, Uppsala, Sweden) according to manufacturer's guidelines. Thereafter, protein concentration was determined using Bio-Rad Protein Assay (Bio-Rad, Hercules, USA).

### RNA-EMSA:

The protein-RNA binding assay and non-denaturing PAGE was carried out based on the protocol published by Stangl and co-workers (Stangl, H., Gruber, F. & Kubicek, C.P. Characterization of the Trichoderma reesei cbh2 promoter. Curr. Genet. 23, 115-22 (1993)). However, the procedure was adapted for adequate separation of 262 nt to 428 nt lncRNA and performed under RNase-free conditions. In order to achieve a correct folding, in vitro synthesized lncRNA was denatured at 95° C. for 5 min and cooled to room temperature immediately before preparing the EMSA reactions. For each EMSA-approach, 1 μg lncRNA was applied in a 10 μl reaction and supplemented with a 0.25 to 8-fold molar excess of Xyr1. 1 μg of lncRNA-QM6a (11.21 μmol), lncRNA-QM9414 (9.82 μmol) and lncRNA-Rut-C30 (6.86 μmol) correspond to 1180 ng, 1030 ng and 720.3 ng Xyr1 (105 kDa), respectively.

Binding was achieved in EMSA buffer by incubation at 22° C. for 10 min. The samples were separated on a 4% native polyacrylamide gel (30:0.36 acrylamide:bis-acrylamide) in 0.5-fold concentrated TBE at 4° C. for 45 min at 160 volt and 15 mAmp per gel. RNA-EMSA gels were analyzed by ethidium bromide staining (1 μg/ml in 0.5-fold concentrated TBE) for 10 min and imaging using a Gel Doc™ XR+ Imaging System with Image Lab™ Software version 5.2 (Bio-rad). The strength of each band was quantified using Image Lab version 5.2 and related to the free lncRNA in the absence of Xyr1.

### Results:

Binding of Xyr1 to the 3 lncRNA versions was analyzed. For all three versions the addition of increasing amounts of Xyr1 results in a shift of the RNA, depending on the Xyr1-concentration. This shift first appeared as a smear moving upwards on the gel, and finally resulted in a clearly defined band at the uppermost position when the highest concentrations of Xyr1 were applied.

This result suggests the formation of an lncRNA-Xyr1 complex due to physical interaction of the two regulatory factors. The ratio of free lncRNA, smeared RNA (smear) and totally shifted lncRNA forming a complex with Xyr1 (lncRNA-Xyr1) based on the quantities relative to the signal of free lncRNA in the absence of Xyr1 is given in the table below.

TABLE 6
RNA-EMSA of lncRNA-QM61 and Xyr1.

| | Relative | Relative | Relative |
|---|---|---|---|
| Molar excess of Xyr1 | quantity | quantity | quantity |
| relative to lncRNA | free lncRNA | smear | lncRNA-Xyr1 |
| — | 1.00 | 0.00 | 0.00 |
| 0.5-fold | 0.96 | 0.00 | 0.00 |
| 1-fold | 0.12 | 0.29 | 0.00 |
| 2-fold | 0.01 | 0.41 | 0.32 |
| 3-fold | 0.02 | 0.26 | 0.57 |
| 4-fold | 0.00 | 0.00 | 0.76 |

TABLE 7
RNA-EMSA of lncRNA-QM9414 and Xyr1.

| | Relative | Relative | Relative |
|---|---|---|---|
| Molar excess of Xyr1 | quantity | quantity | quantity |
| relative to lncRNA | free lncRNA | smear | lncRNA-Xyr1 |
| — | 1.00 | 0.00 | 0.00 |
| 0.5-fold | 0.65 | 0.00 | 0.00 |
| 1-fold | 0.34 | 0.04 | 0.00 |
| 2-fold | 0.08 | 0.32 | 0.00 |
| 3-fold | 0.01 | 0.10 | 0.03 |
| 4-fold | 0.01 | 0.06 | 0.15 |
| 8-fold | 0.00 | 0.00 | 0.53 |

TABLE 8
RNA-EMSA of lncRNA-Rut-C30 and Xyr1.

| | Relative | Relative | Relative |
|---|---|---|---|
| Molar excess of Xyr1 | quantity | quantity | quantity |
| relative to lncRNA | free lncRNA | smear | lncRNA-Xyr1 |
| — | 1.00 | 0.00 | 0.00 |
| 0.5-fold | 0.98 | 0.00 | 0.00 |
| 1-fold | 0.20 | 0.33 | 0.00 |
| 2-fold | 0.00 | 1.76 | 0.00 |
| 3-fold | 0.01 | 1.32 | 1.29 |
| 4-fold | 0.00 | 1.09 | 1.57 |
| 8-fold | 0.00 | 0.29 | 1.57 |

Slight differences could be observed for the different versions of lncRNA. For all versions a shift was first visible when equimolar amounts of Xyr1 were applied. Yet, in the case of lncRNA-QM9414 and lncRNA-Rut-C30, at least an 8-fold molar excess was required to result in in a total shift, whereas for lncRNA-QM6a, a 4-fold molar excess of Xyr1 was sufficient for a complete saturation of the RNA. These findings are in good accordance with the fact that the QM6a lncRNA contains only eight Xyr1-binding sites, while ten or eleven Xyr1-binding sites are present in those of QM9414 and Rut-C30, respectively.

### Example 6: Investigations of the Impact of the Palindromic DNA-Motive XRE in the Xyr1 Promoter as an Element Crucial for the Regulation of Xyr1 Expression, by Reporter Gene Analysis of Wild-Type and XRE-Deleted Pxyr1::goxA

A 12 bp-long, palindromic DNA-motive in the xyr1 promoter (Pxyr1) has been identified as a potential regulatory element of xyr1 expression. It was termed “Xyr1 regulatory element” (XRE) (SEQ ID NO:37). The impact of XRE on the expression of the gene controlled by Pxyr1 was investigated by reporter gene analysis. For this purpose, two different versions of Pxyr1, i.e. the wild-type version and a mutant lacking XRE, were produced and fused to the reporter gene goxA, encoding the Glucose oxidase A from Aspergillus niger: The constructs were introduced into the genome of T. reesei QM6a_Δpyr4 and the GoxA activity of the obtained strains grown on different carbon sources was assayed.

### Plasmid Construction:

For the construction of the plasmids used for the generation of the Pxyr1::goxA strains, the wild-type version of Pxyr1 was PCR amplified from chromosomal DNA of T. reesei QM6a using the primers pxyr1_fw_cfr (SEQ ID NO:38) and pxyr1_rv_bam-nhe (SEQ ID NO:39). Pxyr1 lacking XRE was generated via splicing by overlap-extension (SOE) PCR. First, two overlapping fragments were amplified from chromosomal DNA of T. reesei QM6a using the primers pxyr1_fw_cfr (SEQ ID NO:38) and pxyr1_Apal_rv (fragment 1, SEQ ID NO:40) or pxyr1_Apal_fw (SEQ ID NO:41) and pxyr1_rv_bam-nhe (fragment 2, SEQ ID NO:39). Then fragment 1 and 2 were used as templates for the SOE PCR with the primers pxyr1_fw_cfr (SEQ ID NO:38) and pxyr1_rv_bam-nhe (SEQ ID NO:39), and the final product was extracted from a gel.

Both Pxyr1-variants were purified and blunt-end ligated into pJET1.2 (Thermo Scientific, Waltham, Mass., USA), yielding the respective pJET-Pxyr1 plasmids. The goxA gene from Aspergillus niger was PCR amplified from pLW-WT (Wurleitner, E. et al. Transcriptional regulation of xyn2 in Hypocrea jecorina. Eukaryot. Cell 2, 150-8 (2003)) using the primers goxa_fw_bam (SEQ ID NO:42) and goxa_rv_bcu-nhe (SEQ ID NO:43), also blunt-end cloned into pJET1.2 and released by digestion with BamHI and Nhel.

Subsequently, the resulting goxA fragment was inserted into the different pJET-Pxyr1 plasmids digested with the same enzymes. Finally, the complete promoter-reporter constructs were excised using Bcul and Cfr9l and ligated into a Bcul and Kpn2l-digested pCD-RPyr4T (Derntl, C., Kiesenhofer, D. P., Mach, R. L. & Mach-Aigner, A. R. Novel strategies for genomic manipulation of Trichoderma reesei with the purpose of strain engineering. Appl. Environ. Microbiol. 81, 6314-23 (2015)) vector, yielding the plasmids pMS-repyr4/pxyr1::goxA, pMS-repyr4/pxyr1_804::goxA, pMS-repyr4/pxyr1_606::goxA, pMS-repyr4/pxyr1_497::goxA, pMS-repyr4/pxyr1_372::goxA and pMS-repyr4/pxyr1_ΔXRE::goxA. Escherichia coli strain Top10 (Invitrogen, Life Technologies, Paisley, UK) was used for all cloning procedures. It was maintained on LB supplemented with 100 μg/ml ampicillin or spectinomycin and grown at 37° C. All PCRs were performed applying peqGOLD Pwo DNA polymerase (PEQLAB, Biotechnologie, Erlangen, Germany) according to the manufacturer's instructions.

### Funqal Transformation:

Protoplast transformation of T. reesei for the generation of the Pxyr1::goxA strains was performed as described previously (Gruber F, Visser J, Kubicek CP, de Graaff LH. The development of a heterologous transformation system for the cellulolytic fungus Trichoderma reesei based on a pyrG-negative mutant strain. Curr Genet. 1990; 18:71-76).

80 μg of Notl-digested plasmid DNA (precipitated and resolved in 15 μl sterile dH₂O) were used for transformation of 107 protoplasts (in 200 μl) of QM6a_Δtmus53_Δpyr4. For selection of prototrophy, 500 μl of the transformation reaction mixture were added to 20 ml melted, 50° C. warm MA medium agar containing 1.2 M sorbitol and 1% (w/v) D-glucose. This mixture was poured into sterile petri dishes. After solidification the plates were incubated at 30° C. for 3 to 7 days until colonies were visible.

### Genomic Characterization:

Homokaryotic Pxyr1::goxA strains were generated by three rounds of vegetative spore propagation on selection medium. Extraction of chromosomal DNA for candidate screening was performed as described by Gruber and co-workers (Gruber, F., Visser, J., Kubicek, C.P. & de Graaff, L.H. The development of a heterologous transformation system for the cellulolytic fungus Trichoderma reesei based on a pyrG-negative mutant strain. Curr. Genet. 18, 71-6 (1990)) and according to the adaptions described previously (Derntl C, Kiesenhofer DP, Mach RL, Mach-Aigner AR. Novel Strategies for Genomic Manipulation of Trichoderma reesei with the Purpose of Strain Engineering. Appl Environ Microbiol. 2015; 81:6314-6323.).

For the pxyr1-wt candidates, the integration of the construct was tested by PCR using the primer pairs 5pyr4_fwd3 (SEQ ID NO:28) and pxyr1_rv_bam-nhe (SEQ ID NO:39) (5′ flank of the pyr4 locus, 2621 bp), goxa_fw_Bam (SEQ ID NO:42) and goxa_rv_Bcu-Nhe (SEQ ID NO:43) (goxA gene, 1836 bp) or tpyr4_rev2 (SEQ ID NO:32) and pyr4_3fwd (SEQ ID NO:31) (3′ flank of the pyr4 locus, 1859 bp). The strains pΔXRE were tested by PCR using the primers 5pyr4_fwd3 (SEQ ID NO:28) and pxyr1_rv_bam-nhe (SEQ ID NO:39) (5′ flank of the pyr4 locus, 2621 bp) or goxa_fw_Bam (SEQ ID NO:42) and goxa_rv_Bcu-Nhe (SEQ ID NO:43) (goxA gene, 1836 bp).

For all PCRs, 10 ng of chromosomal DNA were used as a template and GoTaq G2 polymerase (Promega, Madison, Wis., USA) was applied according to the manufacturer's instructions. In Addition, all candidates were tested via Southern blot analysis as described previously (Derntl C, Kiesenhofer DP, Mach RL, Mach-Aigner AR. Novel Strategies for Genomic Manipulation of Trichoderma reesei with the Purpose of Strain Engineering. Appl Environ Microbiol. 2015; 81:6314-6323). 15 μg Sacll digested chromosomal DNA and a biotinylated goxA-specific probe were used, yielding a signal for the Pxyr1::goxA constructs at 1484 bp and a locus-specific signal at 3511 bp.

The transformation yielded three verified strains of pxyr1-wt L1, _2 and_3) and two strains of pΔXRE L2 and_3). The strains were maintained on MA medium agar containing 1% (w/v) D-glucose or MEX at 30° C.

### Carbon Source Replacement:

For the reporter gene analysis of the obtained Pxyr1::goxA strains, carbon source replacement experiments were done. For each strain, the mycelia were pre-cultured in 200 ml of MA medium supplemented with 0.1% peptone and 1% (w/v) glycerol as the sole carbon source on a rotary shaker (180 rpm) at 30° C. for 24 h. A total of 109 conidia per liter (final concentration) were used as the inoculum. Pre-grown mycelia were washed, and equal amounts were resuspended in 20 ml MA medium without carbon source or MA medium containing 1% (w/v) D-glycerol or 1.5 mM sophorose. Samples were taken after 8 h of incubation from two (pΔXRE) or three (pxyr1-wt) biological replicates derived from independently generated strains.

### Glucose Oxidase A (GoxA) Assay:

GoxA assays were performed as described previously (Mach, R. L. et al. Expression of two major chitinase genes of Trichoderma atroviride (T. harzianum P1) is triggered by different regulatory signals. Appl. Environ. Microbiol. 65, 1858-63 (1999)) using ABTS (2,2′-azino-di-(3ethyl-benzthiazoline sulfonate)) (Molekula Ltd., Gillingham, United Kingdom) and horseradish peroxidase (Sigma-Aldrich). GoxA activities calculated in units are means of technical triplicates and two (pΔXRE) or three (pxyr1-wt) biological replicates derived from independently generated strains and were referred to the biomass (dry weight). One unit of enzymatic activity is defined as the amount of enzyme that oxidizes 1 μmol of D-glucose per min at pH 5.8 and 25° C.

TABLE 9
Strains used for this example.

| | | | Selection |
|---|---|---|---|
| Strain | Abbreviation | Candidates | marker |
| QM6a_Δtmus53_Pxyr1::goxA::Tcbh2 | pxyr1-wt | _1, _2, _3 | reconstitution |
| | | | of pyr4 |
| QM6a_Δtmus53_Pxyr1_ΔXRE:goxA ::Tcbh2 | pΔXRE | _1, _2 | reconstitution |
| | | | of pyr4 |

### Results:

The GoxA activities of strains expressing the goxA gene under the control of the wild-type Pxyr1 (i.e. pxyr1-wt) and strains bearing a deletion of XRE in Pxyr1 (i.e. pΔXRE) are shown below. They are given as relative values referred to pxyr1-wt grown on MA medium without carbon source (NCS). For pΔXRE, an approximately 3-fold increase in goxA activity compared to pxyr1-wt is achieved on all carbon sources. This strengthens the hypthesis that XRE is crucial for negative regulation of xyr1 expression.

TABLE 10
NCS: no carbon source; Gly: glycerol; S: sophorose.

| | | GoxA activity relative |
|---|---|---|
| Strain | Carbon source | to pxyr1-wt, NCS |
| pxyr1-wt | NCS | 1.0 |
| pxyr1-wt | Gly | 0.6 |
| pxyr1-wt | S | 0.8 |
| pΔXRE | NCS | 3.9 |
| pΔXRE | Gly | 3.1 |
| pΔXRE | S | 2.8 |

Example 7: EMSA study on the binding of Xyr1 to the DNA-motive XRE in the xyr1 promoter To investigate whether Xyr1 can bind to the DNA-motive XRE on its own promoter (refer to Example 6), an EMSA study using a FAM-labeled 35 bp dsDNA probe composed of the XRE and its adjacent genomic region was performed. For this purpose, Xyr1 was heterologously expressed and purified as described in Example 5.

### Electrophoretic Mobility Shift Assay (EMSA):

The synthetic, 35 bp-long, FAM-labeled oligonucleotide EMSA Pxyr1_fw Pal-FAM (SEQ ID NO:44) and its complementary oligonucleotide EMSA Pxyr1_rev Pal (SEQ ID NO:45) (Sigma-Aldrich, St. Louis, Mo., USA) were annealed by heating at 95° C. and subsequent cooling to room temperature, yielding the labeled ds DNA fragment used as EMSA probe. The protein-DNA binding assay and non-denaturing PAGE was carried out based on the protocol published by Stangl and co-workers (Stangl, H., Gruber, F. & Kubicek, C.P. Characterization of the Trichoderma reesei cbh2 promoter. Curr. Genet. 23, 115-22 (1993)).

33.4 ng of the labeled probe were applied in a 10 μl reaction and supplemented with a 0.5-fold, 2-fold or 8-fold molar excess of heterologously expressed Xyr1. 33.4 ng (1.47 μmol) of the FAM-labeled probe correspond to 154.35 ng Xyr1 (105 kDa). Binding was achieved in EMSA buffer by incubation at 22° C. for 10 min. The samples were separated on a 5.8% native polyacrylamide gel (30:0.36 acrylamide:bis-acrylamide) containing 5.4% glycerol in 0.5-fold concentrated TBE at 4° C. for 75 min at 160 volt and 35 mAmp per gel. Fluorescence and image analysis of the gels was carried out using a ChemiDoc^{T}M MP Imaging System with Image Lab™ Software version 5.2 (Bio-Rad). The strength of each band was quantified using Image Lab version 5.2 and related to the free probe in the absence of Xyr1.

### Results:

In the table below, quantities of the free probe and the shifted probe forming a complex with Xyr1 (probe-Xyr1) relative to the signal of the free probe in the absence of Xyr1 are given. An 8-fold molar excess of Xyr1 completely shifts the XRE-containing probe whereas a 0.5 or 2-fold molar excess of Xyr1 only yields the signal of the free probe. This indicates the formation of a Xyr1-XRE complex. Thus, in general XRE is identified as a hitherto unknown Xyr1 binding site. Moreover, binding of Xyr1 to its own promoter via XRE can be supposed. Considering the facts that XRE was identified as a negative regulatory element of xyr1 expression in Example 6, this finding leads to the conclusion that XRE plays a central role in negative feedback-regulation of Xyr1.

TABLE 11

| Molar excess of Xyr1 | Relative quantity | Relative quantity |
|---|---|---|
| relative to the probe | free probe | probe-Xyr1 |
| — | 1.00 | 0.00 |
| 0.5-fold | 0.99 | 0.00 |
| 2-fold | 1.09 | 0.00 |
| 8-fold | 0.05 | 1.09 |

### Example 8: EMSA Study on the Impact of lncRNA on Binding of Xyr1 to XRE in the Xyr1 Promoter

Similar to Example 7, an EMSA was performed to investigate the impact of the lncRNA on binding of Xyr1 to its own promoter via the newly identified recognition site XRE (see Example 6). Again, as a probe a FAM-labeled 35 bp dsDNA fragment composed of the XRE and its adjacent genomic region on Pxyr1 was used and heterologously expressed Xyr1 was added to shift this probe. The impact of the lncRNA on binding of Xyr1 to XRE was analyzed by the addition of both, Xyr1 and lncRNA-QM6a or lncRNA-Rut-C30 to the XRE-probe in a combined reaction. Heterologous expression of Xyr1 and in vitro synthesis of IcnRNA used for this EMSA study was performed as described in Example 5.

### Electrophoretic Mobility Shift Assay (EMSA):

The synthetic, 35 bp-long, FAM-labeled oligonucleotide EMSA Pxyr1_fw Pal-FAM (SEQ ID NO:44) and its complementary oligonucleotide EMSA Pxyr1_rev Pal (SEQ ID NO:45) (Sigma-Aldrich, St. Louis, Mo., USA) were annealed by heating at 95° C. and subsequent cooling to room temperature, yielding the labeled ds DNA fragment used as EMSA probe. Similarly, in vitro synthesized lncRNA was denatured at 95° C. for 5 min and cooled to room temperature immediately before preparing the EMSA reactions in order to achieve a correct folding. The protein-DNA binding assay and non-denaturing PAGE was carried out based on the protocol published by Stangl and co-workers (Stangl, H., Gruber, F. & Kubicek, C.P. Characterization of the Trichoderma reesei cbh2 promoter. Curr. Genet. 23, 115-22 (1993)). However, the procedure was performed under RNase-free conditions.

33.4 ng of the labeled probe were applied in a 10 μl reaction and supplemented with a 0.5-fold, 2-fold or 8-fold molar excess of heterologously expressed Xyr1. To investigate the impact of lncRNA, a 0.5-fold, 2-fold or 8-fold molar excess of in vitro synthesized IcnRNA was added to the reactions containing 33.4 ng of the FAM-labeled dsDNA probe and an 8-fold molar excess of Xyr1. 33.4 ng (1.47 μmol) of the FAM-labeled probe correspond to 154.35 ng Xyr1 (105 kDa), 131.14 ng lncRNA-QM61 (262 nt) and 314.23 ng lncRNA-Rut-C30 (428 nt).

All nucleic acids were mixed before adding the protein. Binding was achieved in EMSA buffer by incubation at 22° C. for 10 min. The samples were separated on a 5.8% native polyacrylamide gel (30:0.36 acrylamide:bis-acrylamide) containing 5.4% glycerol in 0.5-fold concentrated TBE at 4° C. for 75 min at 160 volt and 35 mAmp per gel. Fluorescence and image analysis of the gels was carried out using a ChemiDoc™ MP Imaging System with Image Lab™ Software version 5.2 (Bio-Rad). The strength of each band was quantified using Image Lab version 5.2 and related to the free probe in the absence of Xyr1.

### Results:

The fluorescence signals resulting from approaches containing either the labeled XRE-probe alone, or together with different amounts of Xyr1 (a 0.5-fold, 2-fold or 8-fold molar excess relative to the probe), or together with an 8-fold molar excess of Xyr1 and increasing amounts of lncRNA-QM61 or lncRNA-Rut-C30 (a 0.5-fold, 2-fold or 8-fold molar excess relative to the probe) were compared. Quantities of the free probe and the shifted probe forming a complex with Xyr1 (probe-Xyr1) relative to the signal of the free probe in the absence of Xyr1 are shown.

As observed for the EMSA presented in Example 7, an 8-fold molar excess of Xyr1 completely shifted the probe containing the XRE of Pxyr1. In the presence of an 8-fold molar excess of lncRNA relative to the probe this shift was completely abolished. Results are shown in the table below.

Binding of lncRNA itself to the XRE has been excluded. Hence, this result suggests that lncRNA competes with the XRE for binding of Xyr1. This is true for both the shorter lncRNA version from QM6a and the longer version from Rut-C30. However, in the case of the QM6a version, a 2-fold molar excess only reduced the shift, while the same concentration of Rut-C30 version led to a complete loss of shift.

This led to the conclusion that the longer lncRNA from Rut-C30 acts as a better competitor than the short one from QM6a. This is in good accordance with the fact that the Rut-C30 lncRNA is a more efficient interaction partner of Xyr1, because it contains three more Xyr1 binding sequences than the shorter QM6a version (see Example 5).

Summed up, this example provides evidence that the lncRNA interferes with the binding of Xyr1 to the XRE on Pxyr1 and, thus, has an impact on the auto-regulation of xyr1 expression.

TABLE 12

| Molar excess of additives | | | | |
|---|---|---|---|---|
| relative to the probe | | | | |
| | lncRNA- | lncRNA- | Relative quantity | Relative quantity |
| Xyr1 | QM6a | Ruc-C30 | free probe | probe-Xyr1 |
| — | — | — | 1.00 | 0.00 |
| 0.5-fold | — | — | 0.94 | 0.00 |
| 2-fold | — | — | 0.78 | 0.00 |
| 8-fold | — | — | 0.04 | 0.37 |
| 8-fold | 0.5-fold | — | 0.02 | 0.46 |
| 8-fold | 2-fold | — | 0.49 | 0.11 |
| 8-fold | 8-fold | — | 0.95 | 0.00 |
| 8-fold | — | 0.5-fold | 0.04 | 0.20 |
| 8-fold | — | 2-fold | 0.80 | 0.00 |
| 8-fold | — | 8-fold | 0.87 | 0.00 |

### Example 9: Production of a T. reeseiQM6a lncRNA-Deletion Strains and Analysis of the Impact of lncRNA on the Expression of Xyr1 Via Reverse Transcription Quantitative PCR (RT-qPCR)

In this study, the impact of lncRNA on the expression of xyr1 was investigated by transcript analysis of xyr1 in a lncRNA-deleted background. For this purpose, a QM6a lncRNA-deletion strain was produced applying the Cre/loxP-system previously published by Steiger and co-workers Steiger, M. G. et al. Transformation system for Hypocrea jecorina (Trichoderma reesei) that favors homologous integration and employs reusable bidirectionally selectable markers. Appl. Environ. Microbiol. 77, 114-21 (2011). This system offers the opportunity to create marker-free deletion strains in a two-step-procedure.

In a first step, a deletion cassette comprising of a marker gene flanked by loxP sites and the upstream- and downstream regions of the target gene was integrated into the genome at the desired locus, thereby deleting the gene of interest. Subsequently, in a second step the intention was to excise the marker by the activity of the Cre recombinase, which causes a recombination event at the loxP sites flanking the marker gene. Hence, the strain QM6a_LoxP carrying the Cre recombinase encoding gene under the control of an inducible promoter integrated at the pyr4-locus was to be used as a recipient strain for transformation of the lncRNA-deletion construct.

However, as the marker gene could not be excised by the activation of the Cre recombinase, the obtained strain AlncRNA_LoxP was used directly for analysis via reverse transcription quantitative PCR (RT-qPCR).

### Plasmid Construction:

The 5′ and 3′ regions flanking lncRNA-Rut-C30 (SEQ ID NO:10) were PCR amplified from chromosomal DNA of T. reesei QM6a and LoxP sites were attached using the primers 5-D for (SEQ ID NO:46) and 5-D rev_LoxP-XmaJI (SEQ ID NO:47) (5′ flank) or 3-D for_LoxP-Xbal-Acc65l (SEQ ID NO:48) and 3-D rev_Ncol (SEQ ID NO:49) (3′ flank). The 1019 bp 5′ flank-fragmentwas extracted from a gel, blunt-end ligated into pJET1.2 (Thermo Scientific, Waltham, Mass., USA) and the appropriate orientation was verified by digest with XmaJI and Kpn2l or XmaJI and Xbal.

In a next step, the 1058 bp 3′ flank-fragment was digested with Ncol and Xbal, extracted from a gel, and subsequently cloned into the pJET-5′ flank vector digested with XmaJI and Xbal. Finally the hph gene (encoding hygromycin B phosphotransferase) was amplified from the vector pRLMex₃₀ (Mach, R. L., Schindler, M. & Kubicek, C.P. Transformation of Trichoderma reesei based on hygromycin B resistance using homologous expression signals. Curr. Genet. 25, 567-70 (1994)) using the primers HygR for_XmaJI (SEQ ID NO:50) and HygR rev_Acc651 (SEQ ID NO:51).

The 2540 bp PCR-product was digested with XmaJI and Acc651, extracted from a gel and inserted into the pJET-5′/3′ flank vector digested with the same enzymes, thereby interrupting the 3′ and 5′ flanking regions of/ncRNA and finally yielding the vector pJET_AlncRNA_5′-hph-3′. For cloning of the construct, Escherichia coli strain Top10 (Invitrogen, Life Technologies, Paisley, UK) was used. It was maintained on LB supplemented with 100 μg/ml ampicillin and grown at 37° C. All PCRs were performed applying peqGOLD Pwo DNA polymerase (PEQLAB, Biotechnologie, Erlangen, Germany) according to the manufacturer's instructions. The final construct was verified by sequencing (Microsynth, Balgach, Switzerland).

### Fungal Transformation:

Protoplast transformation of T. reesei for the generation of the Pxyr1::goxA strains was performed as described previously (Gruber F, Visser J, Kubicek CP, de Graaff LH. The development of a heterologous transformation system for the cellulolytic fungus Trichoderma reesei based on a pyrG-negative mutant strain. Curr Genet. 1990; 18:71-76.). As a template for the fungal transformation, the complete AIncRNA-construct was PCR amplified from pJET_AlncRNA_5′-hph-3′ using the primers 5-D for (SEQ ID NO:46) and 3-D rev_Ncol (SEQ ID NO:49) and extracted from a gel.

The obtained 25 μg template DNA were used for transformation of 107 protoplasts (in 150 μl) of QM6a_Δtmus53_Δpyr4 (LoxP), in the following termed QM6a_LoxP. For selection on the resistance against hygromycin B conferred by the hph gene, 500 μl of the transformation reaction mixture were plated with 10 ml MEX agar containing 1.2 M sorbitol. After regeneration at 30° C. for 4 h, 10 ml overlay media supplemented with 200 μg/ml of hygromycin B (yielding a final concentration of 100 μg/ml per plate) were added. After solidification the plates were incubated at 30° C. for 7 days until colonies were visible.

### Genomic Characterization:

A homokaryotic ΔlncRNA_LoxP strain was generated by six rounds of vegetative spore propagation on selection medium. Extraction of chromosomal DNA for candidate screening was performed as described by Gruber and co-workers (Gruber, F., Visser, J., Kubicek, C.P. & de Graaff, L.H. The development of a heterologous transformation system for the cellulolytic fungus Trichoderma reesei based on a pyrG-negative mutant strain. Curr. Genet. 18, 71-6 (1990)).

The integration of the construct was tested by PCR using the primer pairs HygR for_XmaJI (SEQ ID NO:50) and rev kurz (SEQ ID NO:52) (hph to 3′ flank, 2783 bp), 5-D for (SEQ ID NO:46) and hph 5′ rev (SEQ ID NO:53) (5′ flank to hph, 2447 bp), 5-D for (SEQ ID NO:46) and rev kurz (SEQ ID NO:52) (intact/ncRNA: 1606 bp, ΔlncRNA: 3798 bp) or D_locus 5-up for (SEQ ID NO:54) and HygR 5-rev (SEQ ID NO:55) (locus, 1083 bp). For all PCRs, GoTaq G2 polymerase (Promega, Madison, Wis., USA) was applied according to the manufacturer's instructions. In Addition, the candidate was verified via Southern blot analysis as described previously (Derntl C, Kiesenhofer DP, Mach RL, Mach-Aigner AR. Novel Strategies for Genomic Manipulation of Trichoderma reesei with the Purpose of Strain Engineering. Appl Environ Microbiol. 2015; 81:6314-6323.).

30 μg Ncol digested chromosomal DNA and a biotinylated/ncRNA 5′ locus-specific probe were used, yielding a signal for the parent strain QM6a_LoxP at 1484 bp and a locus-specific signal for ΔlncRNA_LoxP at 1212 bp.

The exclusion of the marker gene as intended based on the protocol published by Steiger and co-workers (Steiger, M. G. et al. Transformation system for Hypocrea jecorina (Trichoderma reesei) that favors homologous integration and employs reusable bidirectionally selectable markers.

Appl. Environ. Microbiol. 77, 114-21 (2011)) was not successful, hence hph is still integrated in the genome of the strain used in this study, replacing the lncRNA-encoding sequence and allowing growth in the presence of hygromycin B. ΔlncRNA_LoxP was maintained on MEX agar containing 5 mM uridine or MEX agar containing 5 mM uridine and 100 μg/ml of hygromycin B at 30° C.

### Transcript Analysis:

For transcript analysis of xyr1 in a lncRNA-deleted background, ΔlncRNA_LoxP and the parent strain QM6a_LoxP were cultivated in MEX containing 1% (w/v) α-D-lactose at 30° C. for 24 h. Fungal mycelia were homogenized in 1 mL of peqGOLDTriFast DNA/RNA/protein purification system reagent (PEQLAB Biotechnologie, Erlangen, Germany) using a FastPrep(R)-24 cell disrupter (MP Biomedicals, Santa Ana, Calif., United States). RNA was isolated according to the manufacturer's instructions, and the concentration was measured using the NanoDrop 1000 (Thermo Scientific, Waltham, Mass., United States). Synthesis of cDNA from mRNA was carried out using the RevertAid™ H Minus First Strand cDNA Synthesis Kit (Thermo Scientific, Waltham, Mass., United States) according to the manufacturer's instructions. The template cDNAs were diluted 1:20. RT qPCRs were performed in a Rotor-Gene Q system (Qiagen, Hilden, Germany). The amplification mixture (final volume 15 μl) contained 7.5 μl 2×iQ SYBR Green Mix (Bio-Rad, Hercules, USA), 100 nM forward and reverse primer, and 2.5 μl cDNA. The primers xyr1f (SEQ ID NO:56) and xyr1r (SEQ ID NO:57) were used for the amplification of xyr1 transcripts.

The analysis was carried out in technical triplicates. The following PCR protocols were followed: 3 min initial denaturation at 95° C., followed by 45 cycles of 15 s at 95° C., 15 s at 60° C. and 15 s at 72° C. (for xyr1 and act) or 3 min initial denaturation at 95° C., followed by 40 cycles of 15 s at 95° C. and 120 s at 64° C. (for sar1). Control reactions and data normalization Cycling conditions using sar1 and act as reference genes and calculations were performed as published previously (Steiger MG, Mach RL, Mach-Aigner AR: An accurate normalization strategy for RT-qPCR in Hypocrea jecorina (Trichoderma reesei). J Biotechnol 2010,145:30-37).

TABLE 13
The strains used for this example.

| Strain | Abbreviation | Strain characteristics |
|---|---|---|
| QM6a_Δtmus53_Δpyr4 (LoxP) | QM6a_LoxP | Uridine auxotrophy due to |
| | | integration of Cre recombinase |
| | | encoding gene at the pyr4-locus |
| QM6a_Δtmus53_Δpyr4 | ΔlncRNA_LoxP | lncRNA deletion, |
| (LoxP)_ΔlncRNA::hph | | Hygromycin resistance, |
| | | Uridine auxotrophy due to |
| | | integration of Cre recombinase |
| | | encoding gene at the pyr4-locus |

### Results:

In the table below, transcript levels of xyr1 produced in the ΔlncRNA_LoxP and in the parental strain QM6a_LoxP are shown. They are given as relative quantities, referred to QM6a_LoxP. The transcript levels of xyr1 produced in the/ncRNA-deleted background in ΔlncRNA_LoxP are significantly lower compared to those in the reference strain carrying the intact/ncRNA gene. This provides evidence that the presence of lncRNA has an enhancing effect on xyr1 expression and supports the conclusion drawn from the EMSA in Example 8 that the lncRNA interferes with the auto-regulation of xyr1 expression by titrating Xyr1.

TABLE 14

| | Transcript levels |
|---|---|
| Strain | relative to QM6a_LoxP |
| QM6a_LoxP | 1.00 |
| ΔlncRNA_LoxP | 0.41 |

### Example 10: EMSA study on the binding of Xyr1 to XRE at the lncRNA locus

Interestingly, BLAST analysis revealed that the palindromic DNA-motive XRE identified in the xyr1 promoter (refer to Example 6) is also present in the lncRNA sequence. It is located shortly downstream of the transcriptional start of lncRNA-Rut-C30, but upstream of lncRNA-QM6a and -QM9414. As a result, XRE is only present in the Rut-C30 lncRNA, whereas it is absent in the shorter versions.

In this regard, a function of XRE in the lncRNA as an additional binding site for Xyr1 might be supposed. This matches to the different impacts observed for the different lncRNA versions. However, in addition to its potential function in the protein-RNA interaction, XRE at the/ncRNA-locus on the fungal genome might play a role in the regulation of the/ncRNA transcription.

To learn whether Xyr1 can bind to XRE in the lncRNA, an EMSA study using a FAM-labeled 35 bp dsDNA probe composed of the XRE and its adjacent genomic region at the/ncRNA-locus was performed similarly to in Example 7. Moreover, as described in Example 8, also the impact of the lncRNA on binding of Xyr1 to XRE at the/ncRNA-locus was investigated. Heterologous expression of Xyr1 and in vitro synthesis of lncRNA used for this EMSA study was performed as described in Example 5.

### Electrophoretic Mobility Shift Assay (EMSA):

The synthetic, 35 bp-long, FAM-labeled oligonucleotide EMSA P_Pal fw_5-FAM (SEQ ID NO:58) and its complementary oligonucleotide EMSA P_Pal rev (SEQ ID NO:59) (Sigma-Aldrich, St. Louis, Mo., USA) were annealed by heating at 95° C. and subsequent cooling to room temperature, yielding the labeled ds DNA fragment used as EMSA probe. Similarly, in vitro synthesized lncRNA RNA was denatured at 95° C. for 5 min and cooled to room temperature immediately before preparing the EMSA reactions in order to achieve a correct folding. The protein-DNA binding assay and non-denaturing PAGE was carried out based on the protocol published by Stangl and co-workers (Stangl, H., Gruber, F. & Kubicek, C.P. Characterization of the Trichoderma reesei cbh2 promoter. Curr. Genet. 23, 115-22 (1993)). However, the procedure was performed under RNase-free conditions.

33.4 ng of the labeled probe were applied in a 10 μl reaction and supplemented with a 0.5-fold, 2-fold or 8-fold molar excess of heterologously expressed Xyr1. To investigate the impact of lncRNA, equimolar amounts or an 8-fold molar excess of in vitro synthesized lncRNA of QM6a or Rut-C30 were added to reactions containing 33.4 ng of the FAM-labeled dsDNA probe and an 8-fold molar excess of Xyr1. 33.4 ng (1.47 μmol) of the FAM-labeled probe correspond to 154.35 ng Xyr1 (105 kDa), 131.14 ng QM6a lncRNA (262 nt) and 314.23 ng Rut-C30 lncRNA (428 nt). All nucleic acids were mixed before adding the protein.

Binding was achieved in EMSA buffer by incubation at 22° C. for 10 min. The samples were separated on a 5.8% native polyacrylamide gel (30:0.36 acrylamide:bis-acrylamide) containing 5.4% glycerol in 0.5-fold concentrated TBE at 4° C. for 75 min at 160 volt and 35 mAmp per gel. Fluorescence and image analysis of the gels was carried out using a ChemiDoc™ MP Imaging System with Image Lab™ Software version 5.2 (Bio-Rad). The strength of each band was quantified using Image Lab version 5.2 and related to the free probe in the absence of Xyr1.

### Results:

The fluorescence signals resulting from approaches containing either the labeled XRE-probe alone, or together with different amounts of Xyr1 (a 0.5-fold, 2-fold or 8-fold molar excess relative to the probe), or together with an 8-fold molar excess of Xyr1 and different amounts of lncRNA from QM6a or Rut-C30 (equimolar amounts or 8-fold molar excess relative to the probe) were compared. Quantities of the free probe and the shifted probe forming a complex with Xyr1 (probe-Xyr1) relative to the signal of the free probe in the absence of Xyr1 are shown.

As observed for the EMSA presented in Example 7, an 8-fold molar excess of Xyr1 completely shifts the probe containing the XRE of the lncRNA, whereas a 2-fold molar excess results in a partial shift and a 0.5-fold molar excess only yields the signal of the free probe.

This suggests that Xyr1 does not only bind to XRE in the xyr1 promoter, but also in to XRE at the/ncRNA-locus. Based on this finding a regulatory impact of Xyr1 on the regulation of/ncRNA transcription via XRE is clearly possible. Moreover, as observed for XRE derived from Pxyr1 (Example 8), both the lncRNA from QM6a and Rut-C30 led to a complete loss of the shift when an 8-fold molar excess relative to the probe was applied.

Equimolar amounts of the QM6a lncRNA in contrast did not affect the complex formation of Xyr1 and XRE, whereas the same concentration of lncRNA from Rut-C30 already reduced the shift. To this end, also in the case of XRE at the/ncRNA-locus, evidence for binding of Xyr1 is provided and different impacts of longer and shorter lncRNA versions on this interaction can be hypothesized.

TABLE 15

| Molar excess of additives | | | | |
|---|---|---|---|---|
| relative to the probe | | | | |
| | lncRNA- | lncRNA- | Relative quantity | Relative quantity |
| Xyr1 | QM6a | Rut-C30 | free probe | probe-Xyr1 |
| — | — | — | 1.00 | 0.00 |
| 0.5-fold | — | — | 1.04 | 0.00 |
| 2-fold | — | — | 0.34 | 0.48 |
| 8-fold | — | — | 0.00 | 0.58 |
| 8-fold | 1-fold | — | 0.05 | 0.60 |
| 8-fold | 8-fold | — | 1.07 | 0.00 |
| 8-fold | — | 1-fold | 0.33 | 0.16 |
| 8-fold | — | 8-fold | 1.12 | 0.00 |

### Example 11: Analysis of the Impact of Xyr1 on the Expression of the lncRNA Locus Via RT-qPCR of T. reesei QM6a_ΔXyr1

In this study, the impact of Xyr1 on the expression of the/ncRNA locus was investigated by transcript analysis of/ncRNA in a xyr1-deleted background. The deletion strain QM6a_Δxyr1 and its parent strain QM6a_Δtmus53 were pre-grown and replaced to different carbon sources, and samples were prepared for transcript analysis via reverse transcription quantitative PCR (RT-qPCR). The quantities of the different lncRNA versions from QM6a, QM9414 and Rut-C30 were determined in separate PCRs.

### Carbon Source Replacement:

Both strains, QM6a_Δxyr1 and QM6a_Δtmus53, were pre-cultured in 250 ml of MA medium supplemented with 0.1% peptone and 1% (w/v) glycerol as the sole carbon source on a rotary shaker (180 rpm) at 30° C. for 24 h. A total of 109 conidia per liter (final concentration) were used as the inoculum. Pre-grown mycelia were washed, and equal amounts were resuspended in 20 ml MA medium without carbon source or MA medium containing 1% (w/v) D-glucose, 0.5 mM D-xylose or 1.5 mM sophorose. Samples were taken after 3 h of incubation.

### Transcript Analysis:

Fungal mycelia derived from the carbon source replacement experiment were homogenized in 1 mL of peqGOLDTriFast DNA/RNA/protein purification system reagent (PEQLAB Biotechnologie, Erlangen, Germany) using a FastPrep(R)-24 cell disrupter (MP Biomedicals, Santa Ana, Calif., United States). RNA was isolated according to the manufacturer's instructions, and the concentration was measured using the NanoDrop 1000 (Thermo Scientific, Waltham, Mass., United States).

Synthesis of cDNA from mRNA was carried out using the RevertAid™ H Minus First Strand cDNA Synthesis Kit (Thermo Scientific, Waltham, Mass., United States) according to the manufacturer's instructions. The template cDNAs were diluted 1:20. RT qPCRs were performed in a Rotor-Gene Q system (Qiagen, Hilden, Germany). The amplification mixture (final volume 15 μl) contained 7.5 μl 2×iQ SYBR Green Mix (Bio-Rad, Hercules, USA), 100 nM forward and reverse primer, and 2.5 μl cDNA. The primers:
up-for_2 (SEQ ID NO:6) and rev_1.Intron (SEQ ID NO:1); QM6a
for qPCR_QM9414 (SEQ ID NO:60) and rev_up-Intron (SEQ ID NO:3); QM9414;
up-for_1 (SEQ ID NO:61) and rev up-Intron (SEQ ID NO:3); Rut-C30.

The analysis was carried out in technical triplicates. The following PCR protocols were followed: 3 min initial denaturation at 95° C., followed by 50 cycles of 15 s at 95° C., 15 s at 60° C. and 20 s at 72° C. (for/ncRNA) or 3 min initial denaturation at 95° C., followed by 40 cycles of 15 s at 95° C. and 120 s at 64° C. (for sar1). Control reactions and data normalization using sar1 as a reference genes and calculations were performed as published previously (Steiger MG, Mach RL, Mach-Aigner AR: An accurate normalization strategy for RT-qPCR in Hypocrea jecorina (Trichoderma reesei). J Biotechnol 2010,145:30-37).

TABLE 16
The strains used for this example.

| Strain | Abbreviation | Source |
|---|---|---|
| QM6a_Δtmus53 | | Steiger et. al., 2011 |
| QM6a_Δtmus53_Δxyr1 | QM6a_Δxyr1 | Mello-de-Sousa et. al., 2015 |

### Results:

In this experiment, the production of/ncRNA transcripts in a xyr1-deleted background was analyzed. The transcript levels resulting from three different RT-qPCRs (QM6a, QM9414 and Rut-C30) of the two strains QM6a_Δxyr1 (xyr1-deleted background) and QM6a_Δtmus53 (parental strain) grown on four different carbon sources (no carbon source, glucose, xylose or sophorose) are shown.

For comparing the transcript levels of lncRNA produced in QM6a_Δxyr1 and QM6a_Δtmus53 depending on the growth condition (carbon source), the data is given as relative quantities referred to NCS (of the respective PCRs and strains). For the wild-type strain QM6a_Δtmus53, the levels of all lncRNA transcripts were significantly higher under cellulase repressing conditions (glucose, G) compared to cultivation in medium without any carbon source (NCS).

In QM6a_Δxyr1, in contrast, the transcript levels of NCS and G are rather equal, hence pointing to a loss of carbon source dependent regulation of/ncRNA transcription in a xyr1-deleted background. Moreover, it should be noted that the transcript levels resulting from the cultivation on sophorose (S) as an inducing condition were slightly reduced in QM6a_Δxyr1 compared to NCS, whereas they were slightly increased in QM6a_Δtmus53. However, the effect was not significant.

TABLE 17
Transcript levels relative to PCR NCS. NCS: no carbon
source; G: glucose; XO: xylose; S: sophorose.

| | | Carbon source | | | |
|---|---|---|---|---|---|
| Strain | PCR lncRNA | NCS | G | XO | S |
| QM6a_Δtmus53 | QM6a | 1.00 | 2.21 | 1.01 | 1.10 |
| QM6a_Δtmus53 | QM9414 | 1.00 | 3.16 | 1.20 | 1.35 |
| QM6a_Δtmus53 | Rut-C30 | 1.00 | 3.73 | 1.14 | 1.49 |
| QM6a_Δxyr1 | QM6a | 1.00 | 1.40 | 1.04 | 0.67 |
| QM6a_Δxyr1 | QM9414 | 1.00 | 1.38 | 1.11 | 0.45 |
| QM6a_Δxyr1 | Rut-C30 | 1.00 | 1.89 | 0.52 | 0.63 |

For comparing the transcript levels of/ncRNA produced in QM6a_Δxyr1 and QM6a_Δtmus53 regarding the ratio of the different/ncRNA versions, the data is given as relative quantities referred to the PCR/ncRNA (of the respective strain and growth condition). In general the transcript levels of longer versions, QM9414 and Rut-C30, are low compared to the transcript levels of the shortest version, QM6a, yet in QM6a_Δxyr1 this effect was conspicuously more pronounced than in QM6a_Δtmus53 on all carbon sources. This suggests a shift of the ratio of longer and shorter lncRNA versions towards the short variant in a xyr1-deleted background.

TABLE 18
Transcript levels relative to the QM6a lncRNA. NCS: no
carbon source; G: glucose; XO: xylose; S: sophorose

| | | PCR lncRNA | | |
|---|---|---|---|---|
| Strain | Carbon source | QM6a | QM9414 | Rut-C30 |
| QM6a_Δtmus53 | NCS | 1.00 | 0.46 | 0.70 |
| QM6a_Δtmus53 | G | 1.00 | 0.72 | 1.22 |
| QM6a_Δtmus53 | XO | 1.00 | 0.55 | 0.75 |
| QM6a_Δtmus53 | S | 1.00 | 0.60 | 0.97 |
| QM6a_Δxyr1 | NCS | 1.00 | 0.26 | 0.40 |
| QM6a_Δxyr1 | G | 1.00 | 0.65 | 1.10 |
| QM6a_Δxyr1 | XO | 1.00 | 0.29 | 0.40 |
| QM6a_Δxyr1 | S | 1.00 | 0.54 | 0.88 |

Summed up, the results presented in table 18 of and this example indicate that Xyr1 acts on the regulation of the/ncRNA locus expression, which is in well accordance with the finding that Xyr1 binds to the XRE at the locus (refer to Example 10).

1-10. (canceled)

11: A filamentous fungal host cell producing a polypeptide of interest, said host cell comprising:
a) at least one polynucleotide encoding the polypeptide of interest;
b) one or more native or heterologous polynucleotide encoding a long non-coding RNA (lncRNA), wherein:
i) said lncRNA comprises more than 262 nucleotides and comprises a contiguous nucleotide sequence at its 3′ end, absent any poly-adenylation tail, that is at least 70% identical to that of SEQ ID NO: 8, SEQ ID NO: 9, SEQ ID NO: 10, SEQ ID NO: 19, SEQ ID NO: 20 or SEQ ID NO: 21; and/or
ii) said lncRNA comprises 2 or more Xyr1 binding sequences;
and wherein said one or more native or heterologous polynucleotide is fused with and operably linked to a heterologous promoter, whereby the one or more polynucleotide is transcribed into the lncRNA.

12: The filamentous fungal host cell of claim 11 which is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes or Trichoderma cell.

13: The filamentous fungal host cell of claim 11 which is an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma Iongibrachiatum, Trichoderma reesei or Trichoderma viride cell.

14: The filamentous fungal host cell of claim 11 which is a Trichoderma reesei cell.

15: The filamentous fungal host cell of claim 11, wherein the polypeptide of interest is native or heterologous to the host cell.

16: The filamentous fungal host cell of claim 11, wherein the polypeptide of interest is a secreted polypeptide.

17: The filamentous fungal host cell of claim 11, wherein the polypeptide of interest is a hormone, enzyme, receptor or portion thereof, antibody or portion thereof.

18: The filamentous fungal host cell of claim 11, wherein the polypeptide of interest is a hydrolase, isomerase, ligase, lyase, oxidoreductase or transferase.

19: The filamentous fungal host cell of claim 11, wherein the polypeptide of interest is an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase or xylanase.

20: The filamentous fungal host cell of claim 11, wherein the lncRNA, absent any poly-adenylation tail, comprises or consists of a nucleotide sequence that is at least 70% identical to that of SEQ ID NO: 8, SEQ ID NO: 9, SEQ ID NO: 10, SEQ ID NO: 19, SEQ ID NO: 20 or SEQ ID NO: 21.

21: The filamentous fungal host cell of claim 11, wherein the lncRNA comprises 3 or more Xyr1 binding sequences; preferably 4, 5, 6, 7, 8, 9, 10, 11, 12, 13, 14, 15, 16, 17, 18, 19 or 20 or more Xyr1 binding sequences.

22: The filamentous fungal host cell of claim 11, wherein the Xyr1 binding sequences comprise one or more XBS having the nucleotide sequence shown in SEQ ID NO: 11, wherein at least one of the three A or T residues may be substituted for another nucleic acid, and/or one or more XRE having the nucleotide sequence shown in SEQ ID NO:37.

23: The filamentous fungal host cell of claim 11, wherein the heterologous promoter is a constitutive or an inducible promoter.

24: The filamentous fungal host cell of claim 11, wherein the heterologous promoter is obtained from the genes for Aspergillus nidulans acetamidase, Aspergillus niger neutral alpha-amylase, Aspergillus niger acid stable alpha-amylase, Aspergillus niger or Aspergillus awamori glucoamylase, Aspergillus oryzae TAKA amylase, Aspergillus oryzae alkaline protease, Aspergillus oryzae triose phosphate isomerase, Fusarium oxysporum trypsin-like protease, Fusarium venenatum amyloglucosidase, Fusarium venenatum Daria, Fusarium venenatum Quinn, Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase Ill, Trichoderma reesei endoglucanase V, Trichoderma reesei xylanase I, Trichoderma reesei xylanase II, Trichoderma reesei xylanase III, Trichoderma reesei beta-xylosidase, or Trichoderma reesei translation elongation factor.

25: The filamentous fungal host cell of claim 11, wherein the heterologous promoter is the Trichoderma reesei bgl1 promoter.

26: A method of producing a polypeptide of interest in a filamentous fungal host, comprising the steps of:
a) cultivating a filamentous fungal host cell as defined in claim 11 under conditions conducive to the production of the polypeptide of interest; and, optionally
b) recovering the polypeptide of interest.

27: A method of improving the production, the productivity and/or the yield of a polypeptide of interest in a filamentous fungal host cell, comprising the steps of:
a) providing a filamentous fungal host cell comprising at least one polynucleotide encoding the polypeptide of interest; and
b) modifying the host cell to comprise one or more native or heterologous polynucleotide encoding a long non-coding RNA (lncRNA), wherein:
i) said lncRNA comprises more than 262 nucleotides and comprises a contiguous nucleotide sequence at its 3′ end, absent any poly-adenylation tail, that is at least 70% identical to that of SEQ ID NO: 8, SEQ ID NO: 9, SEQ ID NO: 10, SEQ ID NO: 19, SEQ ID NO: 20 or SEQ ID NO: 21; and/or
ii) said lncRNA comprises 2 or more Xyr1 binding sequences;
and wherein said one or more polynucleotide is fused with and operably linked to a heterologous promoter, whereby the one or more polynucleotide is transcribed into the lncRNA;
wherein the production, the productivity and/or the yield of the polypeptide of interest is improved in the modified host cell compared to its non-modified parent.
